(19) United States
(12) Patent Application Publication
(10) Pub. No.: US 2025/0009413 A1
(43) Pub. Date: Jan. 9, 2025

(54) INSTRUMENT END EFFECTOR WITH JAW MECHANISM AND MOVEABLE COMPONENT AND RELATED DEVICES, SYSTEMS, AND METHODS

(71) Applicant: INTUITIVE SURGICAL OPERATIONS, INC., Sunnyvale, CA (US)

(72) Inventors: Michael Whitlock, Costa Mesa, CA (US); Adrit Lath, Palo Alto, CA (US); Adam J. Ross, Prospect, CT (US)

(73) Assignee: INTUITIVE SURGICAL OPERATIONS, INC., Sunnyvale, CA (US)

(21) Appl. No.: 18/709,999

(22) PCT Filed: Nov. 9, 2022
(86) PCT No.: PCT/US2022/049404

§ 371 Date: May 14, 2024

Related U.S. Application Data
(60) Provisional application No. 63/279,500, filed on Nov. 15, 2021.

(51) Int. Cl. A61B 18/14; A61B 17/29; A61B 18/00; B25J 9/10

(52) U.S. Cl. CPC A61B 18/1447; B25J 9/106; A61B 2017/2902; A61B 2017/2936; A61B 2017/2939; A61B 2018/00172; A61B 2018/0063; A61B 2018/1455

(57) ABSTRACT

A medical instrument may comprise a shaft (391) and an end effector (350) coupled to a distal end portion of the shaft. The end effector may comprise a jaw mechanism (350), a movable element (380) ranslatable relative to the jaw mechanism, a first actuation element (399) operably coupled to the end effector and translatable relative to the shaft, and a second actuation element (398) operably coupled to the end effector and translatable relative to the shaft. Translation of the first actuation element in a first direction relative to the shaft drives closing of the jaw mechanism. Translation of the second actuation element in a second direction relative to the shaft, opposite from the first direction, drives opening of the jaw mechanism. Translation of the second actuation element relative to the shaft in the first and second directions drives translation of the movable element relative to the jaw mechanism.

### CROSS-REFERENCE TO REPLATED APPLICATION

This application claims priority to U.S. Provisional Application No. 63/279,500 (filed Nov. 15, 2021), titled “INSTRUMENT END EFFECTOR WITH JAW MECHANISM AND MOVEABLE COMPONENT AND RELATED DEVICES, SYSTEMS AND METHODS” the entire contents of which are incorporated by reference herein.

### FIELD

Aspects of this disclosure relate generally to instrument end effectors and related devices, systems, and methods, for example, for use in computer-assisted teleoperated manipulator systems. More specifically, aspects of the disclosure relate to end effectors with a jaw mechanism and another movable component, and remotely-controlled instruments including such end effectors.

### INTRODUCTION

Remotely-controlled instruments generally comprise end effectors, which are often disposed at a distal end portion of the instrument and comprise one or more functional components, such as, for example, a jaw mechanism, a stapler, a knife, a camera, an electrode, a sensor, etc., to perform one or more functions of the instrument, such as cutting, sealing, grasping, imaging, etc. The functions performed by an end effector may be controlled and driven by mechanical forces or other inputs (e.g., electrical energy, illumination, irrigation, etc.) received by the instrument via various interfaces generally located at a proximal end portion of the instrument. In some such instruments, actuation elements run from the proximal end portion along an instrument shaft to transmit forces and/or other functionality from a transmission mechanism at the proximal end portion of the instrument to the end effector. Such remotely-controlled instruments can be manually operated, for example, via one or more manually-actuated inputs at a handle or other interface mounted at the proximal end portion. Alternatively, such remotely-controlled instruments may be coupled to or configured to be coupled to computer-assisted manipulator systems, which may be operably coupled to a remotely located console that provides the interface to receive input from a user.

One type of end effector comprises a jaw mechanism and a movable component configured for translational movement relative to the jaw mechanism, such as a cutting component, a staple firing mechanism, etc. The jaw mechanism comprises jaw members that are pivotable between open and closed configurations, for example to grasp an object and/or perform other operations on the object. While the object is grasped by the jaw mechanism, the movable component can be translated relative to the jaw mechanism to perform some other operation on the grasped object, such as cutting the grasped object, firing staples into the grasped object, etc. The jaw mechanism may also include additional functional elements, such as electrodes for electrosurgical functions. The aforementioned end effectors that have a jaw mechanism and a movable component may be referred to hereinafter as multiple degree of freedom of motion (“multiple-DOF”) end effectors because they have multiple components configured to move in various degrees of freedom relative to other parts of the end effector. For example, one degree of freedom of motion is associated with closing/opening the jaw mechanism and another degree of freedom of motion is associated with translation of the movable component. (The multiple degrees of freedom of motion being referred to above in relation to the term “multiple-DOF end effector” comprise motion of a component of the end effector relative to other components of the end effector, in contrast to degrees of freedom of motion of the entire end effector itself relative to an instrument shaft.) One example of a multiple-DOF end effector is a vessel sealer. In a vessel sealer, the jaw members comprise electrodes for supplying electrosurgical energy to seal a material (e.g., tissue) grasped between the jaw members and the movable element comprises a translating cutting element for cutting the material grasped between the jaw members.

In various applications, the shaft and end effector of an instrument are used in workspaces with relatively limited space. Remotely controlled instruments that utilize a multiple-DOF end effector may include, industrial instruments, medical instruments (e.g., surgical instruments, imaging instruments, diagnostic instruments, therapeutic instruments, etc.), or any other type of tool or instrument. For example, in the context of medical instruments, the workspace may comprise a portion of a patient's body and the end effector and shaft may be inserted into the workspace via an incision or natural orifice. Thus, it is generally desirable to provide instruments, and in particular the end effectors, that are relatively small and able to be used and manipulated within the relatively space-constrained regions found in various applications. For example, if components of an instrument that extend through the shaft, such as actuation elements, can be made to take up less space within the shaft, this may allow for the shaft diameter to be reduced, which may allow for less collateral tissue damage to occur as a result of insertion through the opening (e.g., a smaller incision may be made). As another example, if components of an instrument that extend through the shaft, such as actuation elements, can be made to take up less space within the shaft, this may also allow for additional components to be included within the same size shaft, thus expanding the capabilities of the instrument.

As size of instruments become smaller, however, challenges arise in the ability to accurately and effectively perform functions with the instrument, including instruments that are configured for multiple degrees of freedom motion. This is due to the general need to provide drive forces to actuate the various degrees of freedom motion, which can be challenging in small spaces due to frictional considerations, routing actuation elements through the shaft, transmitting large enough forces via the actuation elements, etc.

Accordingly, a need exists to provide end effectors configured for achieving multiple degree of freedom motion that are relatively small using actuation elements that occupy less space, and/or to otherwise improve performance of instrument end effectors.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with an embodiment, a medical instrument comprises a shaft comprising a proximal end portion and a distal end portion, and an end effector coupled to the distal end portion of the shaft. The end effector comprises a jaw mechanism, a movable element translatable relative to the jaw mechanism, a first actuation element operably coupled to the end effector and translatable relative to the shaft, and a second actuation element operably coupled to the end effector and translatable relative to the shaft. Translation of the first actuation element in a first direction relative to the shaft drives closing of the jaw mechanism. Translation of the second actuation element in a second direction relative to the shaft, opposite from the first direction, drives opening of the jaw mechanism. Translation of the second actuation element relative to the shaft in the first and second directions drives translation of the movable element relative to the jaw mechanism.

In accordance with another embodiment, a medical instrument comprises a shaft comprising a proximal end portion and a distal end portion, and an end effector. The end effector comprises a clevis supported by the distal end portion of the shaft, a jaw mechanism comprising two opposing jaw members pivotably coupled to the clevis, and an actuation link engaged with the jaw members and the clevis. The actuation link is moveable in translation relative to the clevis along a proximal direction to drive pivoting of the jaw members to open the jaw mechanism and in a distal direction to drive pivoting of the jaw members to close the jaw mechanism. The end effector further comprises a movable component moveable in translation relative to the jaw members along the proximal and distal directions. The medical instrument further comprises a first actuation element configured to push the actuation link in the distal direction to drive closing of the jaw mechanism and a second actuation element coupled to the movable component. Within a first range of motion, the second actuation element is translatable relative to the actuation link to drive translation of the movable component. Within a second range of motion, the second actuation element is translatable in the proximal direction relative to the shaft to pull the actuation link in the proximal direction to drive opening of the jaw mechanism.

In accordance with yet another embodiment, a method of operating an instrument comprising a shaft and an end effector coupled to the shaft, comprises closing a jaw mechanism of the end effector by translation of a first actuation element in a first direction relative to the shaft. The method further comprises translating a movable component of the end effector in the first direction over a first distance relative to the jaw mechanism, while the jaw mechanism is in a closed state, by translation of a second actuation element in the first direction relative to the shaft. The method further comprises translating the movable component in a second direction back over the first distance relative to the jaw mechanism, while the jaw mechanism is in the closed state, by translation of the second actuation element in the second direction relative to the shaft, the second direction being opposite the first direction. The method further comprises opening the jaw mechanism by continuing to translate the movable component in the second direction relative to the shaft over a second distance extending in the second direction from the first distance.

In accordance with yet another embodiment, a method of operating a medical instrument comprises closing a jaw mechanism of the medical instrument by translating a first actuation element to push an actuation link of the medical instrument to translate in a first direction. The method further comprises in a closed state of the jaw mechanism, extending or retracting a movable component relative to the jaw mechanism by translating a second actuation element coupled to the moveable component relative to the actuation link within a first range of motion. The method further comprises opening the jaw mechanism by translating the second actuation element in a second direction within a second range of motion to pull the actuation link to trans-late in the second direction.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 is a schematic view of an embodiment of an instrument.

FIG. 2 is a perspective view of an embodiment of an end effector and associated actuation elements of an instrument.

FIG. 3A is a perspective view of the end effector of FIG. 2 in a first configuration.

FIG. 3B is a perspective end view of the end effector of FIG. 2 in a second configuration.

FIG. 4 is a cross-section of the end effector of FIG. 2, with the section taken along 5-5 in FIG. 3A.

FIG. 5 is a perspective view of a movable component and first and second actuation elements, in isolation, of the embodiment of FIG. 2

FIGS. 6A-6E comprise side views of portions of the end effector of FIG. 2 in various states, with the jaw members shown in ghost and the clevis omitted to reveal internal parts. FIG. 6A illustrates the end effector in a first state. FIG. 6B illustrates the end effector in a second state. FIG. 6C illustrates the end effector in a third state. FIG. 6D illustrates the end effector in a fourth state. FIG. 6E illustrates the end effector in a fifth state.

FIG. 7 is a perspective view of another embodiment of an end effector and drive members associated therewith of an instrument.

FIG. 8 is a perspective, detailed view of a portion of the end effector and drive member associated therewith of FIG. 7, with a jaw member shown in ghost and clevis omitted to reveal internal parts.

FIG. 9 is a cross-section of the end effector of FIG. 7, with the section taken along 10-10 in FIG. 7.

FIGS. 10A-10E comprise side views of portions of the end effector and drive member associated therewith of FIG. 7 in various states, with jaw members shown in ghost and other components being omitted to reveal internal parts. FIG. 10A illustrates the end effector in a first state. FIG. 10B illustrates the end effector in a second state. FIG. 10C illustrates the end effector in a third state. FIG. 10D illustrates the end effector in a fourth state. FIG. 10E illustrates the end effector in a fifth state.

FIG. 11 is a schematic view of an embodiment of a computer-assisted medical system.

FIGS. 12A and 12B comprise side views of portions of an embodiment of end effector configured as a stapler, with jaw members shown in ghost and other components being omitted to reveal internal parts. FIG. 12A illustrates the end effector in a first state. FIG. 12B illustrates the end effector in a second state.

FIG. 13 is a cross-section of the end effector of FIG. 12A, with the section taken along 13-13 in FIG. 12A.

### DETAILED DESCRIPTION

As noted above, it can be desirable to reduce the space occupied by components of an instrument, such as the actuation elements that extend through the shaft. This may allow the diameter of the end effector and/or the shaft of the instrument to be reduced, and/or it may allow for additional components to be used within the same sized shaft or end effector. In particular, in some instruments space might be relatively constrained when passing through joints that couple an end effector to a shaft, as the motion of the joints may even further limit the space available for internal components, and therefore reducing the space occupied by actuation elements extending through the joints may allow other components that might not have fit through the joints to now be routed through the joints.

End effectors also can be configured to carry out multiple functions using multiple degrees of freedom of motion. For example, some end effectors may have at least two components that are movable along at least two degrees of freedom of motion, such as, for example, pivoting (opening/closing) of jaw members relative to each other and translating a moveable component (e.g., translating a cutting element) relative to the jaw members. To drive these two differing motions, at least two actuation elements have been used in conventional instrument architectures. In addition, a relatively large amount of force may be needed to close the jaw mechanism, and therefore the actuation element that provides that driving force to the jaw mechanism may need to be relatively robust. Accordingly, the number and size of actuation elements extending to the end effector means that a significant amount of space within the shaft and joints (if present) is occupied, making it challenging to reduce the size of the shaft/joints or to add additional components in the shaft/joints. Furthermore, in instruments in which joints are present, the actuation elements need to be flexible enough to bend along with the joints, which excludes some types of actuation elements (e.g., rigid rods, sheet metal bars, or other actuation elements that provide sufficient rigidity along a longitudinal axis of the actuation element to transmit the force without buckling or bending), which can further complicate the configuration and arrangement of the actuation elements.

Accordingly, embodiments disclosed herein may provide multi-DOF end effectors with corresponding actuation elements that are configured to reduce the amount of space occupied by the actuation elements, while also still being usable in instruments having one or more joints. In some embodiments, multi-DOF end effector is configured such that a jaw closing motion is driven by a pushing motion of a first actuation element in a distal direction, a jaw opening motion is driven by a pulling motion of a second actuation element in a proximal direction, and translation of a movable component (e.g., a cutting element or staple firing mechanism) is driven by both pushing and pulling motions of the second actuation element along distal and proximal directions. (Proximal and distal directions referred to herein are as shown in the Figures).

In some embodiments, the first actuation element comprises a flexible push member (e.g., a push-coil, cut tube, etc.), which is an actuation element of a type that is flexible about its longitudinal axis in multiple degrees of freedom and capable of transmitting pushing forces in an axial direction. In some embodiments, the entirety of the length of the first actuation element is configured as a flexible push member, while in other embodiments part of the first actuation element is configured as a flexible push member and one or more other parts of the first actuation element are configured as relatively more rigid push members that can transfer pushing forces (e.g., a rigid tube, beam, etc.) but which do not exhibit lateral flexibility in multiple directions. Flexible push members are described in greater detail below.

The first actuation element is pushed against an actuation link in the distal direction to drive closure of the jaw mechanism. The jaw closing motion may require a relatively large amount of force, especially when, for example, an object is being compressed between the jaw members, and therefore the first actuation element, which can transfer relatively large forces when being pushed in an axial direction, is well suited for driving the jaw closing motion. The first actuation element can transfer large pushing forces because the flexible push member portion thereof, and the other portions thereof if present, are relatively strong (configured to bear a relatively high load) in axial compression. For example, in embodiments in which the flexible push member comprises a push-coil, when a pushing (compressive) force is applied longitudinally to one end of the push-coil, the push-coil relatively can transfer the load axially because each coil of the push-coil is pressed against adjacent coils and thus the coils cannot move relative to one another in the longitudinal direction. Thus, the coil-pipe under compression acts much like a rod, bar, or rigid tubular member with solid walls, transferring substantially all of the pushing forces applied to one end thereof along the length of coil to an object at the other end of the push-coil. Other types of fully flexible push members may act in a similar fashion. To prevent buckling and enable even stronger pushing forces to be applied, the flexible push member may be constrained laterally. Moreover, unlike a rigid compression member such as a rod, rigid tubular member, bar, etc., the flexible push member is flexible in lateral directions and can bend relatively freely while still delivering strong pushing forces, and thus the flexible push member is well suited for use with instruments that have one or more joints disposed between the end effector and the shaft, as it can pass through the one or more joints and due to its flexibility still permit those joints to articulate.

On the other hand, a flexible push member may not be as well suited for transmitting pulling forces, because they generally are relatively weak in tension. When a pulling (tensioning) force is applied longitudinally to one end of the flexible push member, the tension urges portions thereof (e.g., the coils of a push-coil) to pull apart from one another. Thus, the flexible push member under tension tends to stretch (deform and increase in length), much like a spring under tension. This stretching can be problematic when using a flexible push member to transmit a pulling force. Therefore the present disclosure contemplates various embodiments in which a second actuation element is used to drive the jaw opening motion, which involves pulling the actuation link in a proximal direction. The second actuation element comprises a flexible push-pull member, which is an actuation element of a type that is flexible in multiple lateral directions and capable of transmitting both pulling forces and pushing forces in an axial direction. In some embodiments, a flexible push-pull member may be formed from a flexible member that is relatively strong in tension but less strong in compression (e.g., a cable, wire, filament, or similar) by laterally supporting the flexible member to reduce buckling and increase its strength in axial compression and allow for transmitting compressive loads. In particular, in some embodiments the second actuation element comprises a cable routed through a hollow interior channel within the first actuation element, with the first actuation element laterally supporting the cable. The flexible push-pull member extends distally from a distal end of the first actuation element to extend through an opening of the actuation link to couple with the movable element on a distal side of the actuation link. The flexible push-pull member may be relatively strong (configured to bear a relatively high load) in tension, and thus may be well suited to pulling the actuation link in the proximal direction. The flexible push-pull member may also be strong enough in compression to drive distal motion of the movable component. Within a first range of motion of the second actuation element, the second actuation element can translate freely relative to the actuation link (though the opening in the actuation link) to drive translation of the movable component. Within a second range of motion of the second actuation element, further proximal pulling of the second actuation element causes the movable component, which is attached to the second actuation element, to come into contact with and pull the actuation link proximally. Thus, the second actuation element is able to pull the actuation link proximally to drive the jaw opening motion. The second actuation element is also flexible and can bend relatively freely, and thus is also well suited to pass through the joints of instruments without interfering with articulation of those joints.

Because the second actuation element is concentric with and housed within the first actuation element in some embodiments, the amount of space occupied by the actuation elements is greatly reduced. This allows for the shaft to be made smaller than it otherwise would be in a conventional instrument with two or more actuation elements for operating the end effector that are arranged side-by-side rather than one through another and/or for more components to be routed through the shaft of the same size. Moreover, in various embodiments because both actuation elements are concentric with the shaft and joints (i.e., the actuation elements pass through a central longitudinal axis), a change in length of the actuation elements resulting from articulation of the joints is minimized. Accordingly, a twisted lumen structure for routing the actuation elements through the joints, which may otherwise be used to reduce length change of actuation elements due to actuation of joints, may be omitted, thus reducing the complexity of the instrument, further reducing the diameter of the shaft and/or joints, and/or allowing more room for other components to be routed through the shaft and/or joints.

The description below focusses on a vessel sealer instrument that comprises a jaw mechanism with electrosurgical energy delivery functionality and a translating cutting element as a non-limiting embodiment of a multi-DOF end effector. But those having ordinary skill in the art would appreciate that the present disclosure is not limited to this particular type of end effector and that the principles described below are applicable to other types of multi-DOF end effectors. Another embodiment of such an end effector contemplated by this disclosure is a stapler comprising a jaw mechanism (analogous to the jaw mechanism of the vessel sealer) that grasps an object to be stapled (e.g., tissue) and from which staples are fired into the grasped object. In such a stapler, the movable component is a staple firing mechanism which is translated along one of the jaw members and has a ramp to push staples up out of the jaw member as the staple firing mechanism moves past (and thus has a degree of freedom of motion analogous to the translating cutting element of the vessel sealer). The staple firing mechanism may also comprise a blade, and may cut the grasped object while being translated to fire the staples. In another embodiment, the end effector is configured as a vessel sealer comprising a jaw mechanism with electrosurgical energy delivery functionality (e.g., bipolar electrosurgical sealing functionality) and an extendable/retractable monopolar electrode having additional electrode electrosurgical energy delivery functionality (e.g., monopolar electrosurgical cutting functionality). In such a vessel sealer, the movable component is the extendable/retractable monopolar electrode, which may be translated relative to the jaw mechanism between extended and retracted positions. Other embodiments of multi-DOF end effectors would be apparent to those of ordinary skill in the art in view of the present disclosure and embodiments described herein.

Turning now to the figures, various embodiments are described below in greater detail.

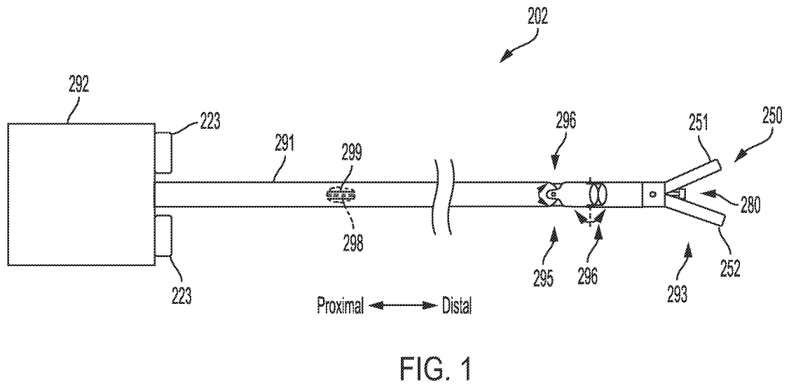

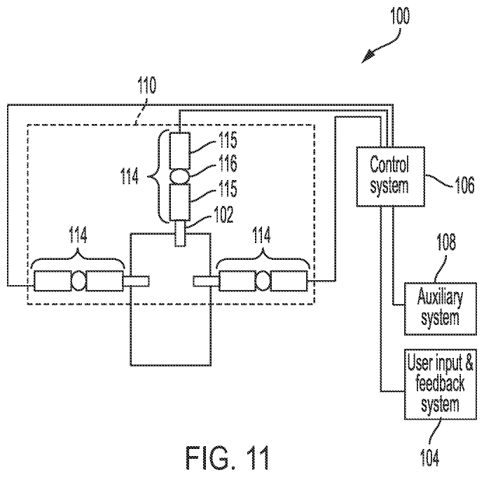

FIG. 1 is a schematic diagram illustrating a side view of an embodiment of an instrument 202. In some embodiments, the instrument 202 may be used and controlled via a computer-controlled system, such as a system 100 described with reference to FIG. 11 below. In other embodiments, the instrument 202 may a manually operable instrument.

As shown in FIG. 1, the instrument 202 comprises a shaft 291 and an end effector 293 attached to the shaft 291 at a distal end portion of the instrument 202, with proximal and distal directions referenced herein illustrated in FIG. 1. The end effector 293 comprises a jaw mechanism 250 and a movable component 280. The instrument 202 also comprises a force transmission system 292, which in some embodiments, is located at a proximal end portion of the instrument 202. The force transmission system 292 is attached to the shaft 291 and comprises one or more drive inputs 223 configured to receive driving forces and/or other inputs that control functions of the instrument 202, such as movements of the instrument 202 (including, e.g., movements of the shaft 291, the end effector 293, and an articulable structure 295) and/or functions of the end effector 293.

The jaw mechanism 250 comprises two jaw members 251, 252 which are coupled to each other to move relative to each other (e.g., by pivoting) between open and closed states. The movable component 280 is translatable distally and proximally relative to the jaw mechanism 250. For example, the end effector 293 may be configured as a tissue sealing instrument (e.g., a vessel sealing instrument), with the jaw members 251 and 252 comprising electrodes to deliver electrosurgical energy (e.g., bipolar electrosurgical energy) to tissue grasped between the jaw members 251 and 252 and with the movable component 280 comprising a cutting element to cut the grasped tissue. In embodiments in which the jaw members 251, 252 comprise electrodes, the electrodes may be formed from the body of the jaw member 251 or 252 itself, such as by different faces or surface features of the jaw member 251 or 252, or the electrodes may be formed as separate parts that are coupled to the body of the jaw members 251 or 252. Electrically conductive power transmission pathways, such as wires, extend through the shaft to the electrodes to electrically couple the electrodes to an electrical power source, such as an electrosurgical unit (ESU), which power source can be coupled to terminals at the force transmission system 292.

The instrument 202 may have various degrees of freedom of motion, which may include internal degrees of freedom of motion of the end effector 293 (e.g., opening and closing of the jaw mechanism 250, translating the movable element 280), as well as degrees of freedom of motion to move the shaft 291 and/or the end effector 293 about the workspace and change the pose of the entire end effector 293 relative to the remainder of the instrument 202. For example, the instrument 202 may comprise one or more articulable structures 295 to allow the end effector 293 to be moved relative to the remainder of the shaft 291. An articulable structure 295 may be any structure that allows for relative motion along one or more degrees of freedom of motion between two components attached to the articulable structure. In some embodiments, an articulable structure 295 may comprise one or more joints 296 arranged to provide the relative motion along the aforementioned one or more degrees of freedom of motion. In an articulable structure 295 comprising multiple joints 295, the joints 296 are coupled directly or indirectly together to form the articulable structure 295. In some embodiments, the degrees of freedom of motion provided by the one or more articulable structures 295 may include pitch, yaw, roll, or any combination thereof of the end effector 293 relative to the shaft 291. As another example of a type of degree of freedom of motion of the instrument 202, the shaft 291 may be rotatable so as to roll relative to the force transmission system 292 or otherwise have degrees of freedom of motion. Moreover, additional optional articulable structures (e.g., joints) (not shown in FIG. 1) may be placed along a length of the shaft 291 to allow the shaft 291 to bend along its length via articulation of one portion of the shaft relative to another portion.

As noted above, the force transmission system 292 may comprise drive inputs 223 that interface with and are driven by the drive outputs of a manipulator system, as described further below with reference to FIG. 11, or they may be driven by manual manipulation such as via various inputs at the force transmission system 292 itself in the form of one or more of a button, trigger, wheel, joystick etc. In addition, the force transmission system 292 may contain various force conversion components (not visible in FIG. 1) to convert the motion of the drive inputs 223 into motion that drives degrees of freedom of the instrument 202. Examples of drive inputs 223 include, but are not limited to, rotational couplers (discs), levers, linear motion inputs/outputs, gears, capstans, pulleys, etc. The forces and motion imparted to the drive inputs 223 may be converted by the force conversion mechanisms of the force transmission system 292 into movement of one or more actuation elements that are operably coupled to the force conversion mechanisms and extend through the shaft 291, with the motion of the actuation elements controlling the degrees of freedom of motion of the instrument 202. Actuation elements can take a variety of forms, such as cables, wires, filaments, rods, rigid tubes, bars, plates, push-coils, etc., or combinations thereof. Depending on the configuration of the actuation element, it may be stronger (configured to bear a relatively higher load) in compression than in tension, stronger in tension than in compression, or roughly equally strong in compression and tension (under the types of loads used in the instrument 202). Thus, some actuation elements may be more suitable for transmitting force from a force transmission system to a moveable component of the instrument through compression (e.g., being pushed), some actuation elements may be more suitable for transmitting force through tension (e.g., being pulled), and some may be suitable for transmitting forces through both tension and compression (e.g., being pushed and pulled). Other actuation elements may transmit force via rotation. In particular, as shown in FIG. 1, the instrument 202 comprises at least a first actuation element 299 and a second actuation element 298. As described above, the first actuation element 299 drives a jaw closing motion of the jaw mechanism 250 by pushing in a distal direction, and the second actuation element 298 drives a jaw opening motion of the jaw mechanism 250 by pulling in a proximal direction. The second actuation element 298 also drives translation of the movable element 280. Additional actuation elements (not illustrated) may extend through the shaft 291 to couple with and drive motion of the joints 296.

In some embodiments, at least a portion of the first actuation element 299 comprises a flexible push member type actuation element. As used herein, a flexible push member is flexible in lateral directions (i.e., about its longitudinal axis) while still being able to transfer pushing forces in the axial direction (i.e., axially rigid) regardless of whether the member is bent. Specifically, in some embodiments, the flexible push member is flexible in at least two orthogonal lateral directions (e.g., pitch and yaw), in contrast to what is referred to herein as a partially flexible push member, which may be flexible in just one lateral direction (e.g., a thin beam). In some embodiments, the flexible push member is flexible in all lateral directions. One example of a flexible push members is a push-coil, which comprises an elongated strip or bar of material (e.g., wire) coiled around and along a longitudinal axis, with adjacent coils being in contact with one another (like a fully compressed spring) to allow for axial transmission of pushing forces. Another example of a flexible push member is a cut tube (e.g., laser cut tube), which comprises a hollow hypo tube that is cut (e.g., by a laser or other means) to form slits through its wall in a specific pattern (e.g., a spiral pattern) that allows the cut tube to flex in lateral directions while still transferring axial pushing forces in a manner similar to a push coil. Coil pipes and some cut tubes may be flexible in all lateral directions. Some examples of coil pipes, cut tubes, and other flexible push members are described in U.S. Patent Application Publication No. 2019/0239967 A1, the contents of which are incorporated herein by reference in their entirety. In some embodiments, only a portion of the first actuation element 299 is configured as the flexible push member, with one or more other portions of the first actuation element 299 being configured as non-flexible or partially flexible push members that can transfer pushing forces in an axial direction but which are not flexible or are flexible in just one lateral direction (e.g., a rigid tube, bar, thin beam, etc.). For example, a first portion that runs through the shaft 291 may be configured as non-flexible or partially flexible member, and this first portion may be coupled to a second portion that runs through the joints 296, with the second portion being configured as a flexible push member (e.g., a coil-pipe) to allow bending through the joints 296. The first actuation element 299 is coupled to an actuation link (not illustrated) of the end effector 293, which the first actuation element 299 pushes on in the distal direction to drive closure of the jaw mechanism 250. The flexible push member portion and the other push member portions (if present) that form the first actuation element 299 can transfer relatively large forces when being pushed and, by positioning the flexible push member portion such that it runs through the joints 296, the first actuation element 299 can be sufficiently flexible to bend relatively freely while passing through the joints 296. The flexible push member portion of the first actuation element 299 may be stronger in pushing than it is in pulling. In other words, it may be stronger in compression than in tension. Fully flexible compression members, such as push-coils, laser-cut tubes, etc., are known in the art, and thus are not described in greater detail herein.

The second actuation element 298 comprises a translating actuation element of a type that can transfer forces by both pushing (compression) and pulling (tension) along a longitudinal direction, while also still being generally flexible in all lateral directions. Such an actuation element that is flexible and can transfer both pushing and pulling forces may be referred to herein as a “flexible push-pull member.” For example, a flexible push-pull member may comprise a flexible member that is relatively strong in tension and relatively weak in compression (when unsupported), such as a cable, wire, filament, or the like, which is surrounded by a supporting structure that laterally supports the flexible member along at least a portion of the length of the flexible member to prevent buckling and thus increase the compressive strength of the flexible member. Thus, laterally supporting the flexible member enables the flexible member to transfer both pulling and pushing forces, and therefore the laterally supported flexible member is one type of a flexible push-pull member. For example, a so-called push-cable or push-pull cable, which comprises a cable surrounded by a supporting structure, is one example of a flexible push-pull member. In some embodiments, the second actuation element 298 may be stronger in tension than in compression, but the second actuation element 298 may nevertheless be sufficiently strong in compression to allow the second actuation element 298 to transfer moderate pushing forces, including pushing forces sufficient to translate the movable component 280 distally and overcome a resisting force, such as may result from resistance of tissue to being cut in the case of cutting element, a resistance of staples to being fired in the case of a staple firing element, etc. In some embodiments, the second actuation element 298 may be roughly equally strong in compression and tension. The second actuation element 298 can translate relative to the actuation link in proximal and distal directions to drive the motion of the movable component 280, and can also pull the actuation link in a proximal direction to drive opening of the jaw mechanism 250. The second actuation element 298 is also flexible and can bend relatively freely while passing through the joints 296.

In some embodiments, the first actuation element 299 forms the lateral support structure for the second actuation element 298, with the second actuation element 298 being routed through a hollow interior channel of the first actuation element 299. The first actuation element 299 may thus act as a guide element and lateral support structure for the second actuation element 298, providing lateral constraining forces that prevent the second actuation element 298 from bending or buckling when being pushed (under moderate forces). In some other embodiments, the second actuation element 298 is routed alongside, rather than through, the first actuation element 299, and a separate supporting structure (e.g., tube, sleeve, coil pipe, etc.) may surround the second actuation element 298 to provide the lateral support to enable the second actuation element 298 to transfer pushing forces.

As noted above, in some embodiments the second actuation element 298 may comprise a flexible member, such as a cable. References herein to cable refer broadly to a tensioning member capable of transmitting tension forces, and also capable of transmitting compression forces when laterally supported. Cables formed from multiple twisted strands of metal are one example of a cable, but it should be understood that “cables” as used herein may also include members formed from non-metals and/or members comprising a single continuous strand (e.g., filament, wire, etc.).

The shaft 291 comprises an outer housing through which various components, such as actuation elements, are routed to transmit force or other functionality to the end effector 293. For example, the housing of the shaft 291 may be shaped as a tube through which one or more passages run to receive and route the various components. The tube may have a central bore and/or bores around a periphery of the tube, such as in a thickness of the housing wall. Components that are routed through the shaft 291 may include, for example, the actuation elements described above to drive movement and/or actuate movement of the instrument 202, electrical power transmission lines, data communication lines, vacuum suction delivery lines, fluid delivery lines, electromagnetic energy delivery lines, etc. As noted above, in some embodiments, the shaft 291 comprises one or more articulable structures 295, and articulation of the articulable structures 295 may also be driven by corresponding actuation elements that are routed through the shaft 291 and transmit force from the force transmission system 292. The end effector 293 is coupled to and supported at a distal portion of the shaft 291, directly or via intermediate parts such as an articulable structure 295. Although one articulable structure 295 is illustrated in FIG. 1, any number of articulable structures 295 may be used. In FIG. 1, the articulable structure 295 is shown in the form of a wrist mechanism comprising two joints 296 configured to provide pitch and/or yaw movement to the end effector relative to the shaft 291. In other embodiments, an articulable structure 295 may be configured differently and may have fewer (e.g., one) or more joints 296 and/or joints 296 that provide different combinations of degrees of freedom of motion.

Although FIG. 1 illustrates the end effector 293 as having a jaw mechanism 250 and a movable element 280, the motion of which represents two degrees of freedom of motion of the end effector, other types of end effectors are contemplated herein that have different components (or different combinations of components) that may move along similar or different degrees of freedom of motion than those of the jaw mechanism 250 and a movable element 280. In particular, in each embodiment contemplated herein, the end effector has at least a first component (e.g., the jaw mechanism 250 in the illustrated embodiment) that moves along a first degree of freedom of motion (e.g., opening/closing) and a second component (e.g., movable component 280) that moves along a second degree of freedom of motion (e.g., translating proximally/distally). Moreover, motion of the first component in a first direction along the first degree of freedom of motion (e.g., closing of jaw mechanism 250) is driven by pushing from the first actuation element 299, motion of the first component in a second direction along the first degree of freedom of motion (e.g., opening of the jaw mechanism 250) is driven by pulling from the second actuation element 298, and motion of the second component along either direction of the second degree of freedom of motion (e.g., distal or proximal translation of the movable component 280) is driven by pushing and pulling of the second actuation element 298.

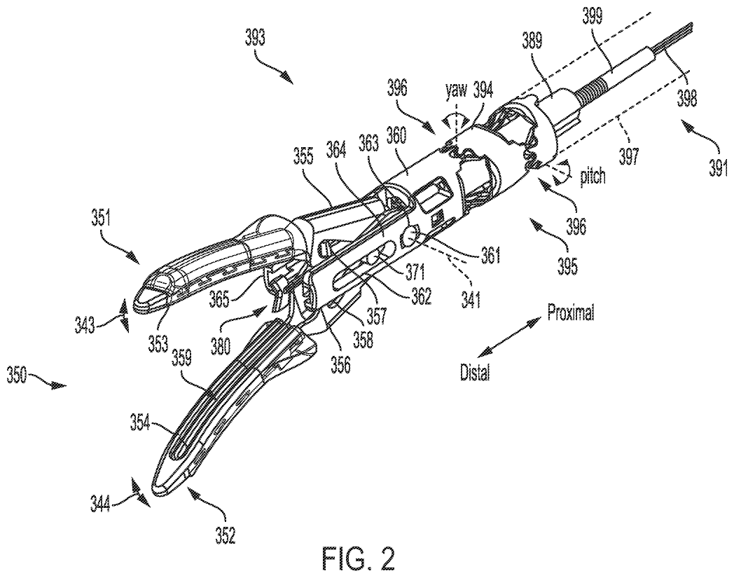

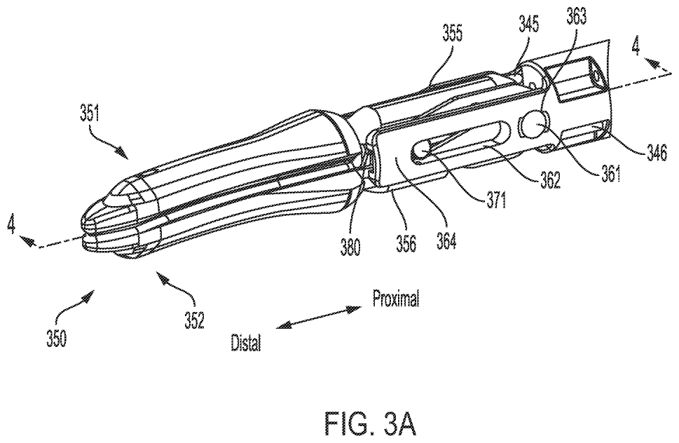

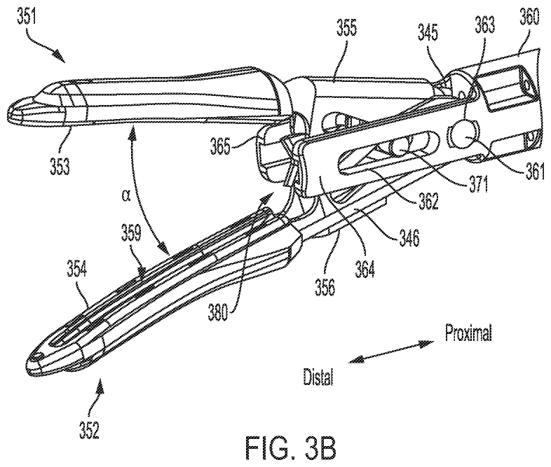

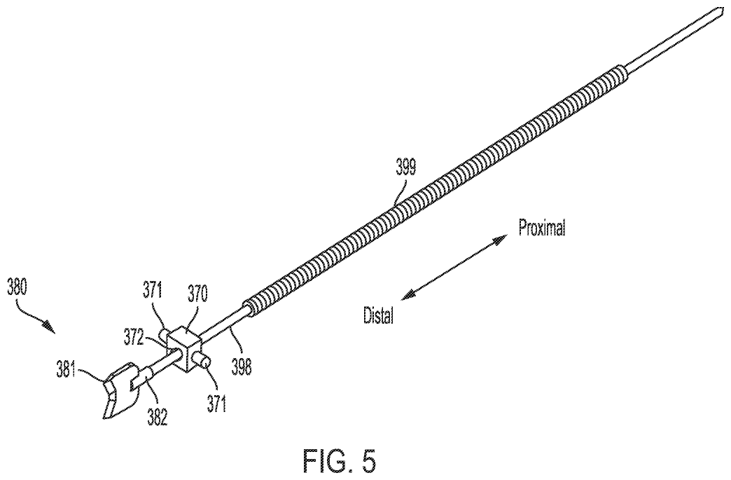

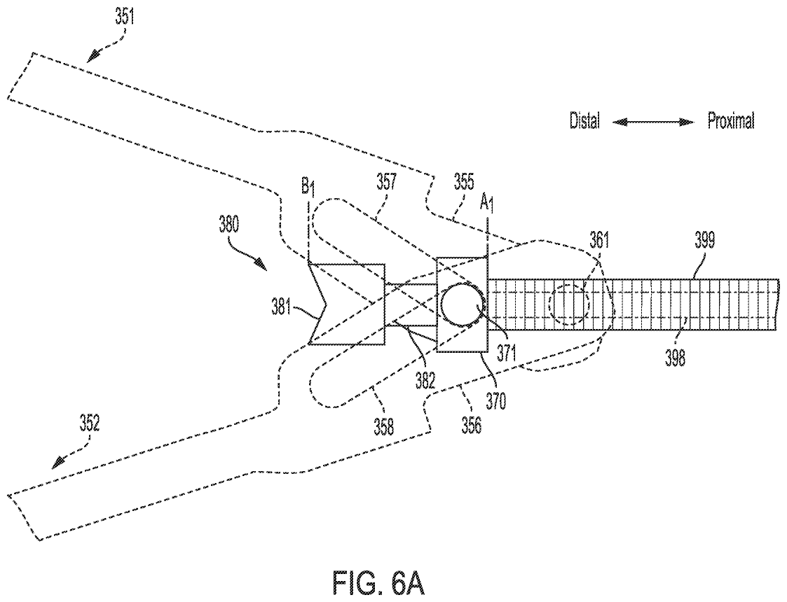

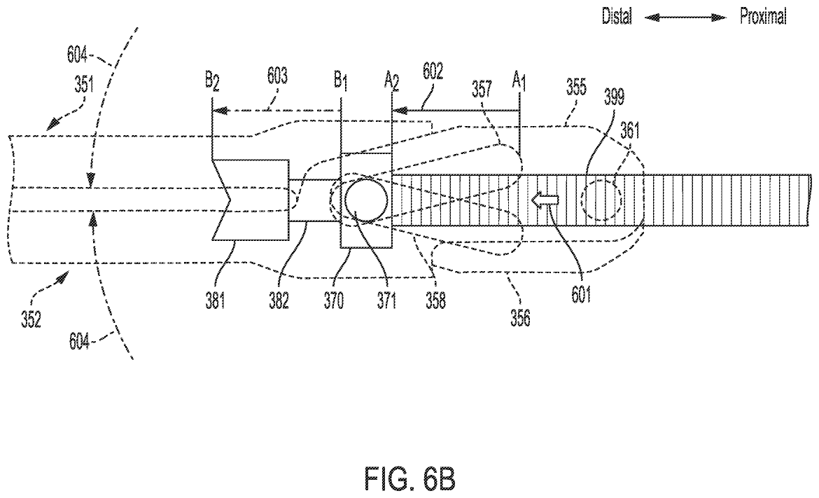

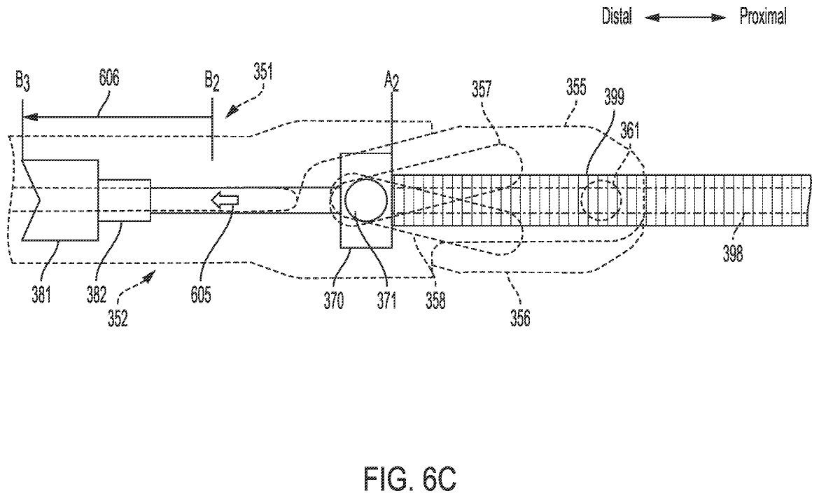

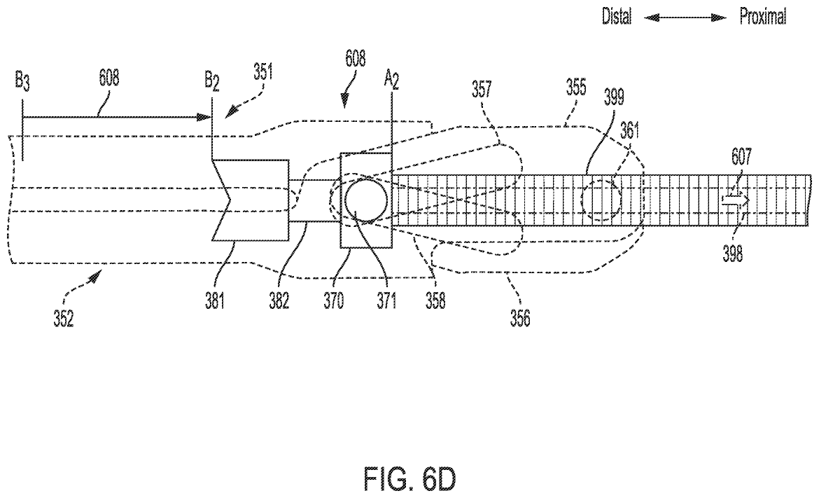

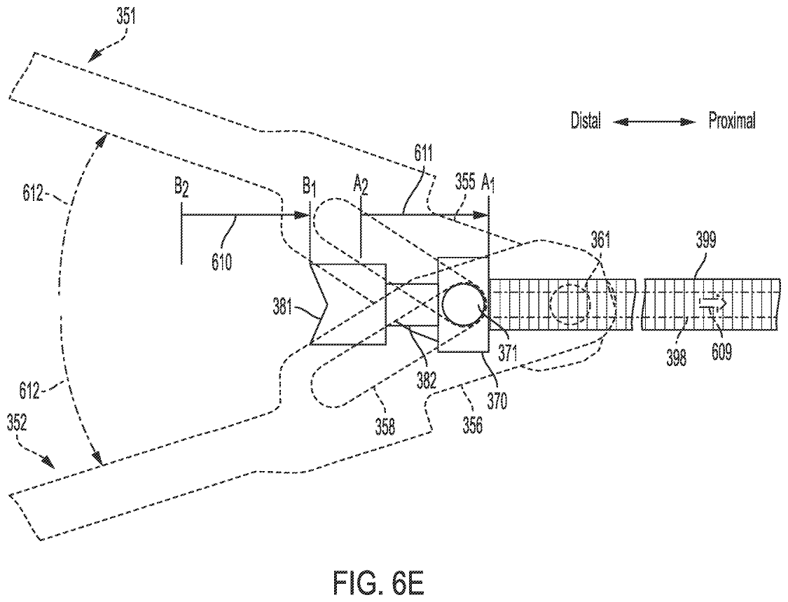

Turning now to FIGS. 2-6E, a first embodiment of an end effector 393 is described in greater detail, as well as associated actuation elements 399 and 398 to drive the end effector 393. The end effector 393 may be used as the end effector 293. Some components of the end effector 393 may be used as components of the end effector 293 described above, and thus the descriptions of the components of the end effector 293 above are applicable to the related components of the end effector 393. These related components are given reference numbers having the same right-most two digits. Although the end effector 393 is one embodiment of the end effector 293, the end effector 293 is not limited to the end effector 393. FIG. 2 comprises a perspective view of the end effector 393, a portion of the shaft 291, and an articulable structure 395 (e.g., a wrist mechanism with two joints 396) coupling the end effector 393 to the shaft 391. FIGS. 3A and 3B are perspective views of the end effector 393 with the jaw mechanism 350 in closed and open states, respectively. FIG. 5 is a cross-section of the end effector 393 with the section taken along 5 in FIG. 3A. FIG. 6 is a perspective view of a movable component 380 and first and second actuation elements 399, 398. FIGS. 6A-6E comprise plan views of a portion of the end effector 393 and actuation elements 398, 399 in various states, with jaw members 351 and 352 depicted as transparent and indicated by dashed lines.

Some parts of the end effector 393 are illustrated in multiple figures. As elements of the end effector 393 are described, one or a few figures which are thought to be particularly pertinent to the aspect will be noted, but it should be understood that other figures besides those that are identified may also illustrate the same part from other perspectives. Thus, the description below will not necessarily describe the figures FIGS. 2-6E separately and in strict sequence.

As shown in FIGS. 2-4, an end effector 393 is coupled to and supported at a distal portion of a shaft 391 via an articulable structure 395, which in the embodiment shown is a wrist mechanism comprising two joints 396 to move the end effector in pitch and yaw relative to the shaft 391. Optionally, the end effector 393 can be coupled directly to the shaft 391 with no articulable structure or may be coupled with an articulable structure providing a single-degree of articulation (e.g., pitch or yaw) or comprising additional joints 396 providing different combinations of degrees of freedom of motion (e.g., pitch, yaw, roll, translation, and/or other articulation type movement). The end effector 393 comprises a jaw mechanism 350 coupled to a clevis 360, which in turn is coupled to the shaft 391 directly or to the articulable structure 396 as shown in FIGS. 2-4. The end effector 393 also comprises a movable component 380. A force transmission system, such as the force transmission system 292 described above, can be coupled at a proximal end portion of the shaft 391 (not shown).

The shaft 391 may house various components, as described above in relation to the shaft 291. In particular, the shaft 391 houses one or more actuation elements, including the actuation elements 399 and 398. The actuation elements transfer motion/forces/torques received from drive inputs at the force transmission system to the end effector 393, articulable structures 395 (e.g., joints 396), and/or other parts of the instrument (such as articulable structures along the shaft) to actuate functions (e.g., closing a jaw mechanism) of the instrument and/or drive other degrees of freedom of motion. For example, the actuation elements may be driven to translate along a longitudinal axis thereof, rotate about the longitudinal axis, or both, with the motion of the actuation elements driving motion of part of the instrument. The actuation elements 399 and 398 in particular are driven to translate along the distal and proximal directions to drive motions of the end effector 293.

Specifically, at least a portion of the first actuation element 399 comprises a flexible push member, such as a push-coil, laser-cut tube, etc. In some embodiments, a first portion of the first actuation element 399 is configured as a flexible push member and a second portion of the first actuation element 399 is configured as a non-flexible or partially-flexible push member, such as a rigid tube, with the first and second portions being coupled together and arranged in sequence along the longitudinal axis of the first actuation element 399. In some embodiments, more than two portions of the first actuation element 399 are configured non-flexible or partially-flexible push members, for example with the flexible push member portion being disposed between two non-flexible or partially-flexible member portions. In some embodiments, the flexible push member portion of the first actuation element 399 may comprise at least a portion that extends through an articulable structure 395, thus enabling the first actuation element 399 to bend with the articulable structure 395. The first actuation element 399 is pushed in the distal direction to drive a jaw closing motion of the jaw mechanism 350, as described above.

The second actuation element 398, on the other hand, comprises a flexible member configured to be capable of transferring both pushing (compressive) and pulling (tensioning) forces, such as a cable or the like that is laterally supported to protect against buckling when pushed (also referred to in the art as a push-pull cable). The second actuation element 398 is pushed in the distal direction to drive an extension motion of the movable component 380 and pulled in the proximal to drive both a retraction motion of the movable component 380 and also a jaw opening motion of the jaw mechanism 350. Specifically, when the second actuation element 398 is translated distally or proximally relative to the shaft 391, over a first range of motion, the motion of the second actuation element 398 drives translation of the movable component 380. Within a second range of motion of the second actuation element 398, proximal translation of the second actuation element 398 relative to the shaft 391 causes the jaw mechanism 350 to open. The configuration and operation of the actuation elements 399 and 398 will be described in greater detail below.

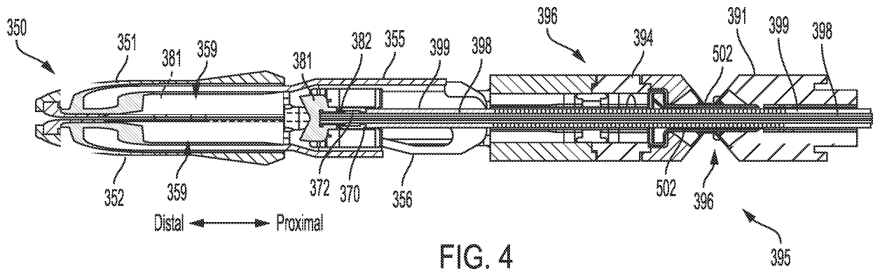

As noted above, in some embodiments the end effector 393 is coupled to the shaft 391 by an articulable structure 395, which in the embodiment shown is a pair of joints 396. In the embodiment illustrated in FIG. 2, two joints 396 are provided that form a wrist mechanism configured to articulate relative to the shaft 391 in pitch and yaw degrees of freedom of motion, as indicated in FIG. 2. The articulable structure 395 may comprise one or more intermediate links 394 that extend between a pair of adjacent joints 396, as shown in FIGS. 3 and 5. The actuation elements 399 and 398 extend through interior channels within the articulable structure 395, as illustrated in FIG. 4, and the actuation elements 399 and 398 may be configured to bend with the 395 as it articulates. Additional actuation elements (not illustrated), such as cables, may extend along (e.g., through) the shaft 391 and may be coupled to articulatable structure (e.g., opposing parts of the links 394) to drive motion of the joints 396 and thus the articulable structure 395, as would be familiar to those of ordinary skill in the art.

The shaft 391 comprises one or more housing structures, including an outer housing 397 as shown in FIG. 2, which is a tube that houses other components of the shaft 391, such as the actuation elements 398 and 399. The outer housing 387 of the shaft 391 is indicated by dashed lines and made transparent in the Figures to allow components inside the shaft 391 to be seen. As shown in FIG. 3, the shaft 391 also comprises an actuation element cover 389, through which the actuation elements 398 and 399 are routed along a portion of the shaft 391. In particular, in some embodiments the actuation element cover 389 may be provided at least at locations where the actuation elements 398 and 399 may be exposed to an exterior environment, such as where the actuation elements 398 and 399 pass through the articulable structure 395. The actuation element cover 389 may thus form a barrier around the actuation elements 398 and 399 to shield them from an external environment. The actuation element cover 389 may also serve to guide the actuation elements 398 and 399 through the joints 396, and may also laterally support the actuation elements 398 and 399 to help resist buckling when the articulable structure 395 articulates. As would be familiar to those of ordinary skill in the art, additional structures could be included in the shaft 391, which are not illustrated herein to avoid obscuring other components. Moreover, in some embodiments the actuation element cover 389 may be omitted.

As shown in FIG. 4, in some embodiments, the articulable structure 395 may comprise blow out plates 502 at the joints 396. Two blow out plates 502 may be positioned on opposite sides of the actuation element 399 as it passes through a given joint 396. The blow out plates 502 may be secured to the shaft 391 and/or the intermediate link 394 around the joint 396 such that the blow out plate 502 flexes as the joint 396 moves. The blow out plates 502 may laterally support the actuation element 399 when the joint 396 is bent, thus prevent the actuation element 399 from blowing out or buckling in the joint. In some embodiments, two blow output plates 502 are provided per joint 296 on opposite sides thereof, with each blow output plate 502 being positioned to reduce buckling along a corresponding directions of motion of the joint 296. In some embodiments, only a single blow out plate 502 is provide for a given joint, for example in situations in which blow out is of concern on only one side of the joint 296. In some embodiments, more than two blow out plates 502 are provide for a given joint. In some embodiments, the blow out plates 502 may be provided in addition to the actuation element cover 389. In some embodiments, the blow out plates 502 may be provided in lieu of to the actuation element cover 389. In FIG. 4, only two blow out plates 502 for one joint 396 are visible due to the perspective of the figure, but in practice there may be two addition plates 502 for the other joint 396. In embodiments with more or fewer joints 296 or differently configured joints, the number and location of the blow out plates 502 may vary.

As noted above, the end effector 393 comprises a jaw mechanism 350. The jaw mechanism 350 may be used as the jaw mechanism 250 described above. The jaw mechanism 350 comprises two jaw members 351, 352 (see FIGS. 2-4), which are also referred to herein as a first jaw member 351 and a second jaw member 352. The jaw members 351, 352 comprise a distal working portion and a proximal end portion, with the proximal end portion being coupled to the clevis 360. The jaw members 351, 352 are arranged in opposition to one another and are pivotable about an axis 341 relative to the clevis 360 about their proximal end portions, thereby opening or closing the jaw mechanism 350. FIG. 3A illustrates the jaw mechanism 350 in a closed state, and FIG. 3B illustrates the jaw mechanism 350 in an open state. In the closed state, the jaw members 351, 352 may be positioned with a relatively small gap (or no gap) between the jaw members 351, 352 and an angle between the jaw members 351, 352 may be relatively small (or may equal zero). In the open state, the distal end portions of the first and second jaw members 351 and 352 are relatively more distant from one another and an angle between the jaw members 351, 352 is relatively larger, for example more than 15 degrees in some embodiments. In various embodiments, the jaw members 351, 352 may be configured to pivot relative to each other over an angle α ranging from about zero degrees (closed state of the jaws) to about 45 degrees.

More specifically, as shown in FIGS. 2-3B, the first jaw member 351 has a proximal end portion 355 that is pivotably coupled to the clevis 360 by pivot pins 361, which engage corresponding holes 363 or recesses in the side walls of the clevis 360 and corresponding holes (not illustrated) in the proximal end portion 355 such that the first jaw member 351 can pivot relative to the clevis 360 about an axis 341. The axis 341 (see FIG. 2) may be referred to herein as a “pivot axis.” The pivot axis 341 is perpendicular to and intersects a longitudinal axis of the clevis 360, which is parallel to a longitudinal axis of the shaft 391 when the articulable structure 395 is not in an articulated state. Pivoting of the first jaw member 351 about the pivot axis 341 causes the distal end of the first jaw member 351 to move toward or away from the second jaw member 352 along an arc as indicated by the arrows 343 in FIG. 2. Similarly, as shown in FIGS. 2-3B, the second jaw member 352 also has a proximal end portion 356 that is pivotably coupled to the clevis 360 by another pivot pin 361 such that the second jaw member 352 can pivot relative to the clevis 360 about the axis 341. Pivoting of the second jaw member 352 about the pivot axis 341 causes the distal end of the second jaw member 352 to move toward or away from the first jaw member 351 along an arc as indicated by the arrows 344 in FIG. 2. In other embodiments (not illustrated), one of the jaw members 351, 352 may be stationary relative to the clevis 360 and the other may pivot relative to the clevis 360 to drive opening and closing of the jaw mechanism 350.

Only one pivot pin 361 is visible in the FIGS. 2-6E, but in practice there may be two pivot pins 361 aligned with one another along the pivot axis 341 and spaced apart from one another along the pivot axis 341 such that a central longitudinal axis of the clevis 360 runs between the two pivot pins 361. One pivot pin 361 is coupled to one side wall 364 of the clevis 360 via a hole 363 or recess in the side wall 364, while another pivot pin 361 (not visible) is coupled to another side wall 365, opposite from the side wall 364, via a hole or recess (not visible) in the side wall 365. This arrangement of the pivot pins 361 leaves an unobstructed space around the central longitudinal axis through which the first and second actuation elements 399 and 398 extend. In some embodiments, the pivot pins 361 may be fixedly coupled to one of the jaw members 351 and 352, while being pivotable relative to the other one of the jaw members 351 and 352.

The jaw members 351 and 352 are driven to pivot about the pivot axis 341 by motion of an actuation link 370, which is coupled to the jaw members 351, 352, as shown in and FIGS. 2-3B and 6A-6E. The actuation link 370 comprises two pins 371 extending in opposite lateral directions perpendicular to the longitudinal axes of the second actuation element 398, as shown in FIG. 5, with each pin 371 engaging a corresponding guide slot 362 in one of the side walls 364 and 365 of the clevis 360 as shown in FIGS. 2-3B. The guide slot 362 constrains motion of the actuation link 370 to motion only along the proximal and distal directions (i.e., motion along the longitudinal axis of the clevis 360). The pins 371 of the actuation link 370 are also respectively engaged with ramped slots 357 and 358 in the proximal end portions 355 and 356 of the jaw members 351 and 352, as shown in FIG. 6A (see also FIGS. 2-3B and 6B-6E). The engagement between the pins 371 and the ramped slots 357 and 358 is such that translation of the actuation link 370 in the distal and proximal directions drives pivoting motion of jaw members 351 and 352.

As noted above, the end effector 393 also comprises a movable component 380, which may be used as the movable component 280. In the embodiment illustrated in FIGS. 2-6E, the movable component 380 comprises a cutting element to cut tissue (e.g., a vessel) or other object grasped between and by the jaw members 351 and 352, and thus the movable component 380 may also be referred to herein as cutting element 380. As shown in FIGS. 4 and 5, the cutting element 380 comprises a blade 381 and an attachment portion 382 to attach the blade 381 to the distal end portion of the second actuation element 398. The attachment portion 382 may be, for example, a cable crimp, or solder, or any other mechanism to attach the blade 381 to the second actuation element 398. As shown in FIGS. 4 and 5, the second actuation element 398 extends from within the first actuation element 399 on a proximal side of the actuation link 370 through the actuation link 370 via a hole 372 in the actuation link 370 to couple with the attachment portion 382 on a distal side of the actuation link 370.

The actuation link 370 is driven to translate relative to the clevis 360 along the proximal and distal directions by the actuation elements 398 and 399. Specifically, the first actuation element 399 is configured to drive translation of the actuation link 370 in the distal direction, thereby driving a jaw closing motion of the jaw mechanism 350. As shown in FIG. 6A (see also FIGS. 4 and 5), the first actuation element 399 is positioned with its distal end adjacent to a proximal side of the actuation link 370. The actuation link 370 has a hole 372, through which the second actuation element 398 extends, but the diameter of the hole 372 is smaller than a diameter of the first actuation element 399, and therefore when the first actuation element 399 is moved in the distal direction it abuts against and pushes the actuation link 370 in the distal direction.

The second actuation element 398, on the other hand, is configured to drive translation of the actuation link 370 in the proximal direction, thereby driving a jaw opening motion of the jaw mechanism 350. Although the second actuation element 398 is not fixedly coupled to the actuation link 370 and is generally free to translate relative to the actuation link 370 through the hole 372, the smallest inner diameter of the hole 372 is smaller than a proximal face of the movable component 380 (specifically, smaller than a diameter of the attachment portion 382 in the illustrated embodiment), and therefore the movable component 380 cannot pass through the hole 372. Accordingly, if the second actuation element 398 is pulled sufficiently far in the proximal direction, a portion of the movable component 380 (e.g., the attachment portion 382) will eventually engage with the actuation link 370 and continued pulling on the second actuation element 398 will in turn force the actuation link 370 to move in the proximal direction, thereby driving the jaw opening motion.

In addition to driving the jaw opening motion, translation of the second actuation element 398 can also drive translation of the moveable component 380 comprising the blade 381. The moveable component 380 is translatable between a first retracted position (a proximal position), a second retracted position (an intermediate position), and an extended position (a distal position). The first retracted position is illustrated in FIGS. 3B and 6A. The second retracted position is shown in FIGS. 4 and 6B. The extended position is shown in FIGS. 4 and 6C, with the extended position being shown in dashed lines in FIG. 4. Because the second actuation element 398 extends through the hole 372 in the actuation link 370 and is not fixedly coupled to the actuation link 370, the second actuation element 398 can freely translate in the distal direction relative to the actuation link 370. Moreover, the second actuation element 398 can also translate in the proximal direction relative to the actuation link 370 between the extended and second retracted positions, until the movable component 380 comes into contact with the actuation link 370 at the second retracted position. Once the movable component 380 has reached the second retracted position and comes into contact with the actuation link 370, further translation of the second actuation element 398 in the proximal direction relative to the shaft 391 results in the movable component 380 pulling the actuation link 370 distally along with the second actuation element 398 (as described above in relation to the jaw opening motion).

Thus, there is a first range of motion of the cable 398 (i.e., between the extended and second retracted positions) within which the second actuation element 398 can freely translate relative to the actuation link 370 in distal and proximal directions as a result of the second actuation element 398 being driven to translate relative to the shaft 391, and there is a second range of motion (i.e., between the second retracted and first retracted positions) in which the second actuation element 398 cannot translate proximally relative to the actuation link 370. Instead, within the second range or motion, proximal translation of the actuation element 398 relative to the shaft 391 causes the actuation link 370 to move proximally along with the cable 398, thereby driving the jaw opening motion. Said another way, within the second range of motion, the actuation element 398 can translate relative to the actuation link 370 in a distal direction, while it translates with the actuation link 370 in a proximal direction.

The jaw members 351 and/or 352 may comprise a track 359 in the form of a trough or channel, as shown in FIGS. 2, 3B, and 4, which receives and guides the blade 381 of the moveable element 380 as the moveable element 380 is extended and retracted while the jaw mechanism 350 is in the closed state. FIG. 4 illustrates the blade 381 in solid lines in the second retracted position and illustrates the blade 381 in dashed lines in the extended position, with the blade 381 in the extended position being received within two opposing tracks 359 of the jaw members 351 and 352 in the closed state.

As noted above, in the embodiment illustrated in FIG. 2 the end effector 393 is configured as part of an electrosurgical vessel sealer instrument, and therefore one or both of the jaw members 351, 352 also comprises an electrode for delivering electrosurgical energy to an object grasped between the jaw members 351 and 352. In the illustrated embodiment, the end effector 393 is configured for bipolar electrosurgical functions, and thus both jaw members 351 and 352 comprise a corresponding electrode 353 and 354, respectively. In other embodiments, end effector 393 may be configured for monopolar electrosurgical functions, and thus only one of the jaw members 351 and 352 may have an electrode. In other embodiments, the end effector 393 may be capable of both bipolar and monopolar electrosurgical functions, and thus both of the jaw members 351 and 352 may have corresponding electrodes, with at least one of the electrodes being selectively activatable and deactivatable to change between a bipolar mode, in which both electrodes are active and electrically coupled to a power circuit, and a monopolar mode, in which the one of the electrodes is deactivated (i.e., disconnected from the power circuit). Returning to the embodiment illustrated in FIG. 2, the electrodes 353 and 354 are each electrically coupled to a corresponding electrical energy transmission line, which are ultimately coupled with an electrical energy supply unit during operation. FIGS. 3A and 3B illustrate an embodiment with dedicated electrical energy transmission lines (e.g., which may be wires, cables, or the like) 345 and 346 for supplying electrical energy to the electrodes 353 and 354, respectively. The electrical energy transmission lines 345 and 346 may extend through the shaft 391, into and through an interior of the clevis 360, and then through an internal channel within and/or along an exterior of the jaw members 351 and 352, respectively, to couple with the electrodes 353 and 354, respectively. In some embodiments, a portion of one or more of the electrical energy transmission lines may be formed from a portion of the instrument that is electrically conductive but which also performs some other functional and is not solely dedicated for electrical energy transmission, such as a housing element of the shaft or an actuation element.

The opening and closing of the jaw mechanism and the extending and retracting of the movable component described above are described below in greater detail with reference to FIGS. 6A-6E, which illustrate a sequence of states that the end effector 393 can be placed in.

FIG. 6A illustrates the end effector 393 in a first state, in which the jaw mechanism 350 is open. In this state the actuation link 370 is located at its most proximal position, indicated by A1 in the figure. Thus, in this state the pins 371 are located near the proximal end portions of the ramped slots 357 and 358, and therefore the jaw members 351 and 352 are located in an open state. Moreover, in the embodiment illustrated in FIG. 6A, in this state the moveable component 380 is located at a first retracted position, indicated by B1 in the figure. In this state, tissue (or other material) may be positioned between the jaw members 351, 352, to allow grasping of the tissue (or other material) when the jaw mechanism 350 is subsequently closed.

In some embodiments, the first state described above and illustrated in FIG. 6A is an initial or default state of the end effector 393, and thus the positions A1 and B1 of the actuation link 370 and movable component 380, respectively, may be considered as the initial, default, or home positions of those components in such embodiments. However, in other embodiments, the initial or default state of the end effector 393 may be different. In some embodiments, an initial or default state of the end effector 393 may be a state in which the jaw mechanism 350 is closed, such as the state illustrate in FIG. 6D. In embodiments in which the initial or default state is one in which the jaw mechanism 350 is closed, the jaw mechanism 350 may be transitioned from this initial state to the open state illustrated in FIG. 6A to allow tissue to be positioned between the jaw members 351 and 352. Thus, in such embodiments, the state illustrated in FIG. 6A may be a second state, rather than a first state, in a sequence of states associated with operation of the end effector 393. The transition from the closed state to the state shown in FIG. 6A is described in greater detail below with reference to FIG. 6E.

In still other embodiments, the initial or default state may be a state (not illustrated) in which the jaw mechanism 350 is open, but in which the movable element is 380 is at a second retracted position B₂, described below. This state is essentially the same as the state illustrated in FIG. 6A, except that the moveable component 380 may be located at a second retracted position B₂, which is shown in FIG. 6B, during this state, instead of in the first retracted position B₁.

FIG. 6B illustrates a second state, in which the jaw mechanism 350 is closed. This state is reached from the first state of FIG. 6A as a result of the first actuation element 399 having been driven to translate in the distal direction, as indicated by the arrow 601. The moving of the first actuation element 399 in the distal direction causes the first actuation element 399 to abut and push the actuation link 370 to also move in the distal direction from the proximal position A₁ to a more distal position A₂. As a result of this motion of the actuation link 370 in the distal direction, the pins 371 move distally along the ramped slots 357 and 358, thus forcing the jaw members 351 and 352 to move towards one another as indicated by the arrows 604. Thus, the distal translation of the first actuation element 399 drives closing of the jaw mechanism 350. In other words, the transition from the first state to the second state corresponds to the jaw closing motion described above, and this jaw closing motion is driven by the first actuation element 399 translating (i.e., being pushed) in the distal direction.

In those embodiments in which the moveable component 380 is at the first retracted position B₁ in the first state, such as the embodiment illustrated in FIG. 6A-B, the moveable component 380 is also caused to translate distally from the first retracted position B₁ to a second retracted position B₂ during the transition from the first state to the second state illustrated in FIG. 6B, as indicated by the arrow 603. This motion of the movable component 380 may be driven primarily by the first actuation element 399, primarily by the second actuation element 398, or by a combination of both, as described in greater detail below.

In some of these embodiments, the moveable component 380 is driven to move from position B₁ to position B₂ entirely by forces transferred by the first actuation element 399 to the actuation link 370 and from the actuation link 370 to the moveable component 380. In other words, in these embodiments the second actuation element 398 is not driven to move (although it is allowed to move) while the first actuation element 399 is being pushed in the distal direction; instead the second actuation element 398 is passively dragged along in the distal direction by the actuation link 370 as a result of the actively driven translation of the first actuation element 399. The second actuation element 398 may resist being dragged along distally by the first actuation element 399, for example, because the drive inputs and drive outputs coupled to the second actuation element 398 resist being moved, but the forces applied to the first actuation element 399 may be sufficiently strong to overcome this resistance.

In other embodiments, the second actuation element 398 is driven to translate distally before and/or while the first actuation element 399 is being driven to move distally, and therefore the movement of the moveable component 380 from position B₁ to position B₂ may be primarily or at least in part driven by the active translation of the second actuation element 398. In some of these embodiments in which the second actuation element 398 is actively driven to help drives movement of the moveable component 380 from position B₁ to position B₂, the second actuation element 398 may begin moving distally prior to the first actuation element 399 beginning to move distally to avoid creating the aforementioned resistance to the distal translation of the actuation link 370.

Regardless of whether motion of the moveable component 380 from B₁ to B₂ is driven primarily by the first actuation element 399, primarily by the second actuation element 398, or some combination thereof, in each of the embodiments it is the first actuation element 399 alone that drives the motion of the actuation link 370 in the distal direction, and hence the jaw closing motion. Because the second actuation element 398 is not fixedly attached to the actuation link 370 and passes through the hole 372, the second actuation element 398 is essentially free to translate distally relative to the actuation link 370. Thus, when the moveable component 380 is moved from B₁ to B₂, even if the second actuation element 398 is driving that motion of the moveable component 380, the motion of the second actuation element 398 does not impart any forces to aid in the motion of the actuation link 370 other than some small frictional forces, but these friction forces are negligible compared to the overall driving forces of the first actuation element 399 and thus are not considered as contributing to the driving of the actuation link 370 for purposes of this disclosure.

In those embodiments (not illustrated) in which the moveable component 380 is already at the second retracted position B₂ in the initial state of the end effector 393, then when transitioning from the first state to the second state, the moveable component 380 may remain stationary while the actuation link 370 is moved.

FIG. 6C illustrates a third state in which the jaw mechanism 350 is closed and the moveable component 380 is extended. This state is reached from the second state of FIG. 6B as a result of the second actuation element 398 being driven to move in the distal direction, as indicated by the arrow 605. This distal motion of the second actuation element 398 causes the moveable component 380 attached to the second actuation element 398 to translate distally, for example, from the second retracted position B₂ to an extended position B₃, as indicated by the arrow 606. This translation of the moveable component 380 distally from the second retracted position B₂ to the extended position B₃ may be referred to herein as firing (or extending) the moveable component 380. Firing the movable component 380 may be part of a cutting operation in the embodiment shown in view of the blade 381 being moved to come into contact with and cut tissue that is grasped between the jaw members 351 and 352 as a result of the jaw closing motion described above.

In the state of FIG. 6C, the actuation link 370 remains at the distal position A₂ while the moveable component 380 is moved distally to the extended position B₃, with the second actuation element 398 being translatable relative to the actuation link 370 during this motion. Specifically, the first range of motion in which the second actuation element 398 is movable in translation along its longitudinal axis relative to the actuation link 370 corresponds to range between the moveable component's 380 positions B₂ and B₃. Thus, during the firing (extending) of the moveable component 380, the second actuation element 398 is within the first range of motion. The first actuation element 399 does not participate in the firing (extending) of the moveable component 380.

FIG. 6D illustrates a fourth state in which the jaw mechanism 350 is closed and the moveable component 380 has been retracted back to the second retracted state. This state is reached from the third state of FIG. 6C as a result of the second actuation element 398 being driven to move in the proximal direction, as indicated by the dash-lined arrow 607. This proximal motion of the second actuation element 398 causes the moveable element 380 attached to the second actuation element 398 to translate proximally from the extended position B₃ back to the second retracted position B₂, as indicated by the arrow 608. This translation of the moveable component 380 from the extended position B₃ to the second retracted position B₂ may also be referred to as retracting the moveable component 380. As with the firing (extending), the retracting of the moveable component 380 is driven entirely by the motion of the second actuation element 398. The first actuation element 399 may be held stationary during this process. As noted above with respect to firing (extending) of the moveable component 380, while the moveable component 380 is being retracted the second actuation element 398 is within the first range of motion, and thus able to translate relative to the actuation link 370.

FIG. 6E illustrates a fifth state in which the jaw mechanism 350 is open. This state is reached from the fourth state of FIG. 6D as a result of the second actuation element 398 being driven to move even further in the proximal direction, as indicated by the dash lined arrow 609. The proximal motion of the second actuation element 398 causes the moveable element 380 to move proximally from the second retracted position B₂ to the first retracted position B₁, as indicated by the arrow 610. During this motion, a portion of the moveable component 380, specifically the attachment portion 382 in the illustrated embodiment, comes into contact with the actuation link 370, and because this prevents further proximal motion of the second actuation element 398 relative to the actuation link 370, the actuation link 370 is pulled along proximally with the retraction of the moveable component 380 as the second actuation element 398 continues to move proximally. Thus, the proximal motion 609 of the second actuation element 398 pulls the actuation link 370 from the distal position A₂ to the proximal position A₁ as indicated by the arrow 611. The distal motion of the actuation link 370 results in the pins 371 sliding proximally along the ramped slots 375 and 358, and thus the jaw members 351 and 352 are moved apart to open positions, as indicated by the arrows 612. Thus, the jaw opening motion is driven by the second actuation element 398 being pulled in the proximal direction from the second retracted position B₂ to the first retracted position B₁. The range of motion of the second actuation element 398 illustrated between fourth state in FIG. 6D and the fifth state illustrated in FIG. 6E corresponds to the second range of motion described above.

While the second actuation element is 398 is being pulled in the proximal direction in the second range of motion to drive the jaw opening motion, the first actuation element 399 also translates proximally to allow the actuation link 370 to move proximally. In some embodiments, the first actuation element 399 is not actively driven to move proximally in this stage of motion, but instead is simply allowed to be pulled along proximally by the motion of the actuation link 370, which is driven entirely by the motion of the second actuation element 398. In other embodiments, the first actuation element 399 is actively driven by the force transmission system to move proximally along with (or prior to) the second actuation element 398, thus avoiding the need for the second actuation element 398 to pull the first actuation element 399 along with the actuation link 370.

In some of embodiments, the first actuation element 399 is not attached to the actuation link 370. Thus, if the first actuation element 399 is driven to move proximally along with the second actuation element 398, the first actuation element 399 does not actually transfer any forces to the actuation link 370. The first actuation element 399 may be driven to move proximally prior to or at the same time as the second actuation element 398.

In some embodiments, the first actuation element 399 is attached to the actuation link 370. Thus, if the first actuation element 399 is driven to move proximally while the second actuation element 398 is also being driven to pull the actuation link 370 proximally, the proximal motion of the first actuation element 399 may apply some pulling force to the actuation link 370, in addition to the pulling force supplied by the second actuation element 398. However, because the first actuation element 399 may be relatively weak at pulling, the pulling forces transferred to the actuation link 370 from the first actuation element 399 may be relatively small compared to the pulling forces transmitted to the actuation link 370 by the second actuation element 398.

Regardless of whether the first actuation element 399 is driven to move during the jaw opening mechanism or whether first actuation element 399 it is attached to the actuation link 370, it is the second actuation element 398 that primarily transmits the force to drive the jaw opening motion. As noted above, even in embodiments in which the first actuation element 399 is attached to the actuation link 370 and driven to move proximally with the second actuation element 398, first actuation element 399 contributes a relatively small proportion of the overall force that is exerted to drive the proximal motion of the actuation link 370, with the second actuation element 398 supplying the predominant proportion of the driving force. Thus, the second actuation element 398 may be considered as the primary driver of the jaw opening motion for purposes of this disclosure. The small amount of force that may be contributed by the first actuation element 399 in some embodiments is not sufficient in and of itself to “drive” the jaw opening motion and thus cannot be considered to be the primary or predominant force driving the motion of the actuation link 370; instead the first actuation element 399 may be regarded as assisting the second actuation element 398.

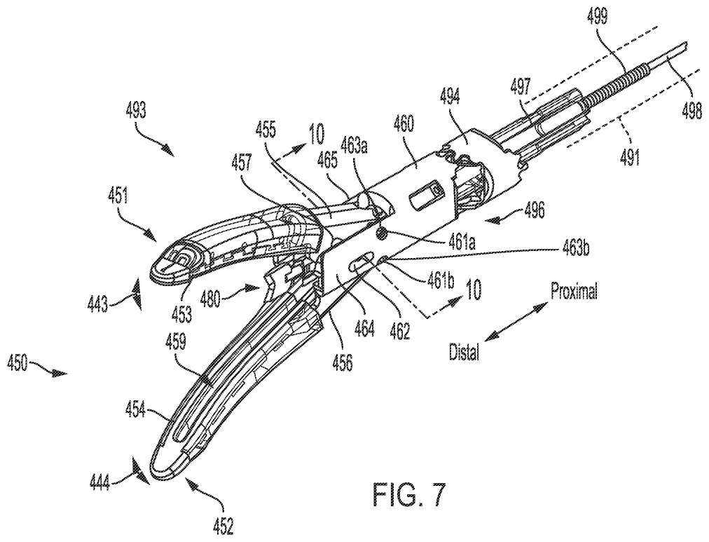

In those embodiments (not illustrated) in which the moveable element 380 is at the second retracted position B₂ during the initial state, after the state illustrated in FIG. 6E the second actuation element 398 may be driven to move the moveable component 380 distally from the first retracted position B₁ to the second retracted position B₂ so as to reset the end effector 393 to the initial state. However, in embodiments such as the illustrated embodiment in which the moveable component 380 is at the first retracted position B₁ in the first state, then the moveable component 380 may be left where it is located in the fifth state—in other words, in such embodiments the fifth state of FIG. 7E reached after performing the series of operations described above may serve as the first state for a subsequent performance of the series of operations. In embodiments in which the initial state of the end effector 393 comprises a closed state of the jaw mechanism 350, then after the state illustrated in FIG. 6E the jaw mechanism 350 may be closed (as described above in relation to FIG. 6B) to return the end effector 393 to the initial state.

In the progression of states described above, with the instrument being embodied as a vessel sealer, an electrosurgical sealing operation may also be performed on an object (e.g., vessel) grasped by the jaw mechanism 350. In particular, the electrosurgical sealing operation may be performed in the second state illustrated in FIG. 6B after closing the jaw mechanism 350 and prior to firing (extending) the blade 381 to cut the grasped object, or the electrosurgical sealing operation may be performed in the fourth state illustrated in FIG. 6D after the blade 381 has been fired to cut the grasped object. The electrosurgical sealing operation may comprise supplying electrosurgical energy (e.g., electricity at a specific voltage and frequency) may be delivered to the electrodes 353 and 354 to cause electricity to flow between the electrodes 353 and 354, with the electricity passing through the tissue grasped by the jaw mechanism in the closed state as it flows between the electrodes 354 and 353. The sealing function may be effectuated by controlling the mode of the electrical energy delivered to the electrode(s) of the end effector, such as the voltage, current, frequency, and duty cycle at which the electrical power is applied, such that the amount of heat generated by the electricity is in the range that would induce coagulation. Electrosurgical functions would be familiar to those of ordinary skill in the art, and thus are not described in further detail herein.

Turning now to FIGS. 7-10E, a second embodiment of an end effector 493 is described in greater detail. The end effector 493 may be used as the end effector 293. The end effector 493 is similar to the end effector 393, with some differences as noted below and shown in the Figures. Parts of the end effector 493 that are similar to parts of the end effector 393 are referred to using reference numerals having the same right-most digits (for example, jaw member 351 and jaw member 451 are similar parts of the end effector 393 and the end effector 493, respectively). The description of the components of the end effector 393 above is applicable to the similar components of the end effector 493, mutatis mutandis, and therefore duplicative descriptions of these similar components of the end effector 493 are omitted below.

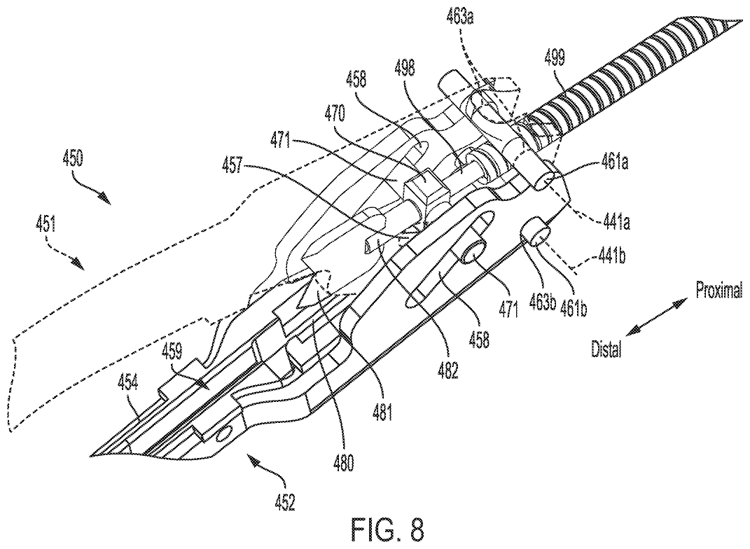

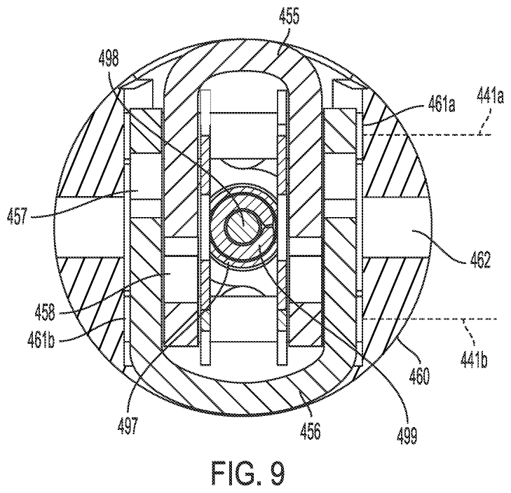

One difference between the end effector 393 and the end effector 493 is that in the end effector 493 there are two separate pivot axes 441a and 441b about which the jaw members 451 and 452 respectively pivot, rather than both jaw members pivoting about the same pivot axis 341 as in end effector 393. As shown in FIGS. 7-9, the first jaw member 451 is pivotably coupled to the clevis 460 by pivot pin 461a extending through the proximal portion 455 of the first jaw member 451 and is pivotable about a pivot axis 441a, while the second jaw member 452 is pivotably coupled to the clevis 460 by pivot pin 461b extending through the proximal portion 456 of the second jaw member and is pivotable about the pivot axis 441b. As shown in FIGS. 8 and 9, the pivot pins 461a and 461b, and hence the pivot axes 441a and 441b, are spaced apart from one another on opposite sides of a longitudinal axis of the clevis 460 and of the second actuation element 498 that drives the moveable component 480. The pivot axes 441a and 441b may be parallel to one another in some embodiments. As shown in FIGS. 7 and 8, the pivot pins 461a and 461b are coupled to the clevis 460 by corresponding holes or recess 463a and 463b, respectively, in both of the side walls 464 and 465 of the clevis 460. As shown in FIGS. 8 and 9, the second actuation element 498 and/or first actuation element 499 may extend along a central longitudinal axis of the clevis 460 through an open space between the pivot pins 461a and 461b.

In some embodiments, including the embodiment illustrated in FIGS. 7-9, the pivot pins 461a and 461b may differ from the pivot pins 361 in that each of the pivot pins 461a and 461b extends fully across the width of the clevis 460 with one end thereof coupling with side wall 464 and the other end coupling with side wall 465, while in an embodiment of the end effector 393 each of the pivot pins 361 extends only partially across the width of the clevis 360 and each couples with just one of the side walls 364 or 365. In the end effector 493, the pivot pins 461a and 461b are able to extend fully across the width of the clevis 460 without interfering with the actuation elements 498, 499 because the pivot pins 461a and 461b are spaced from one another along two spaced apart pivot axes 441a and 441b, respectively, on opposite sides of the actuation elements 498, 499, thus leaving an unobstructed space around the central longitudinal axis, as shown in FIG. 9. In contrast, in an embodiment of the end effector 393 in which the pins 361 are aligned with one another along the pivot axis 341 and the pivot axis 341 intersects with the central longitudinal axis of the clevis 360, the pins 361 cannot extend fully through the width of the clevis 360 without interfering with the actuation elements 398, 399 (at least not without providing some sort of workaround feature to bypass the pins 361).

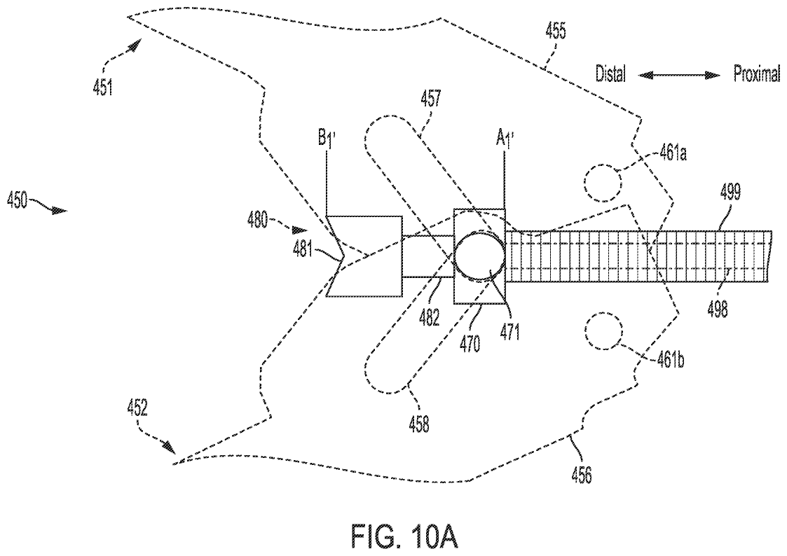

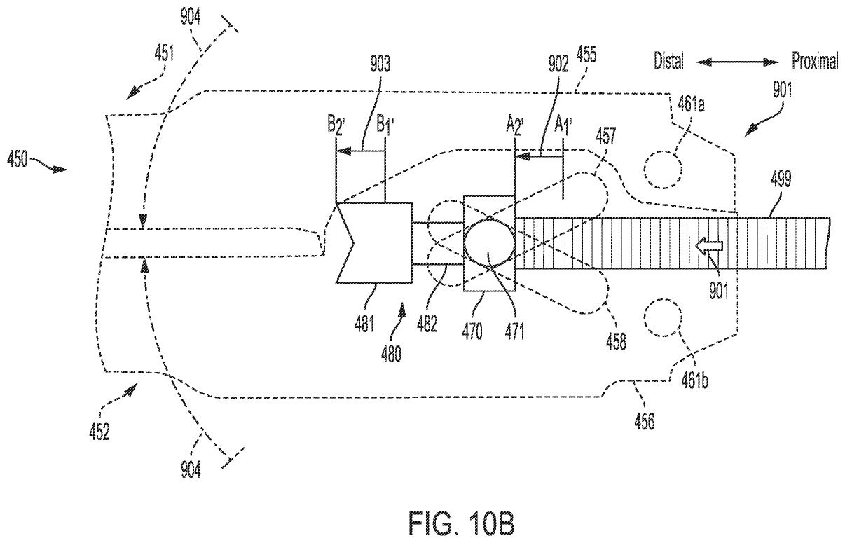

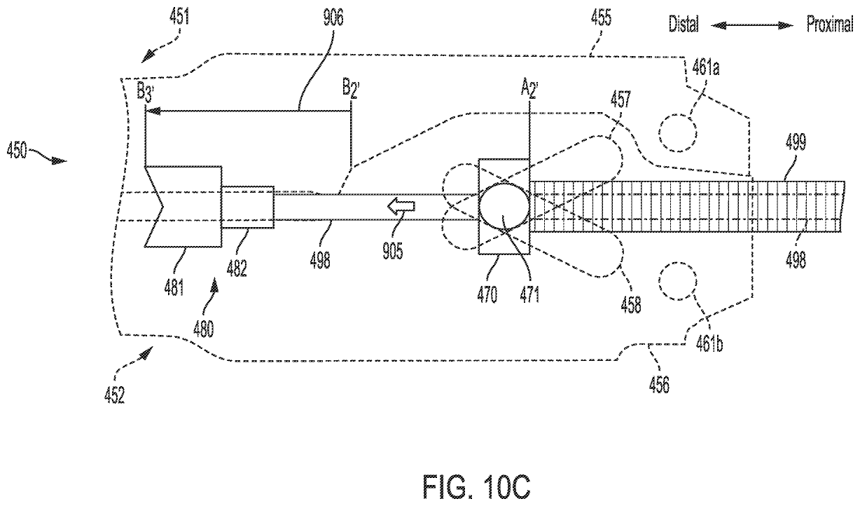

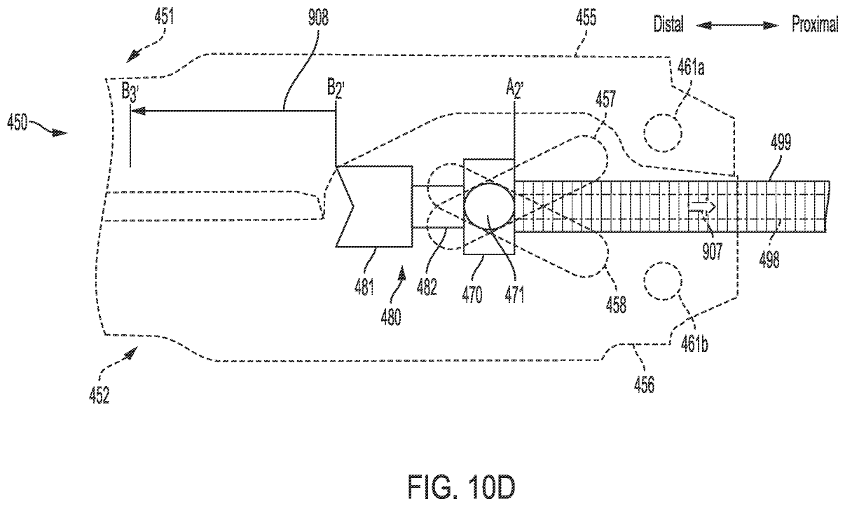

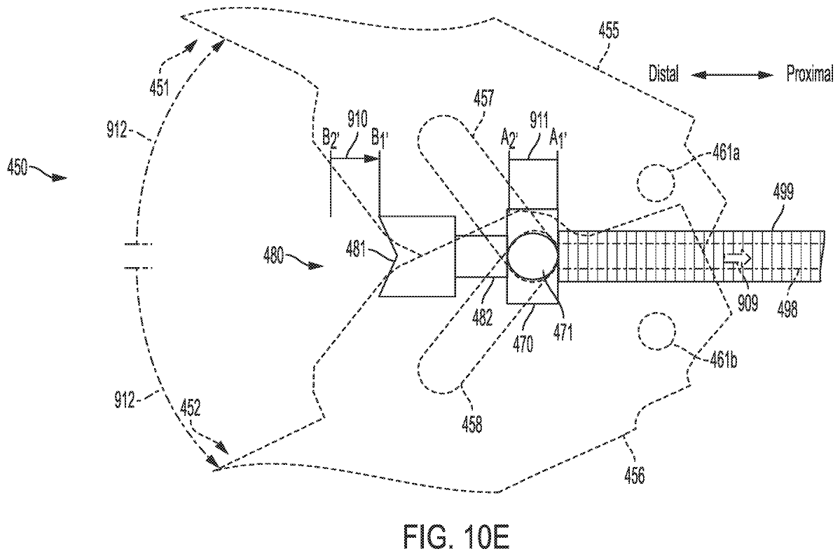

In some embodiments, the clevis 460 of the end effector 493 may have a larger diameter than the clevis 360 of the end effector 393, as a result of the pivot pins 461a and 461b being vertically spaced apart from one another. However, one benefit of the arrangement of the end effector 493 may be that a length of travel of the actuation link 470 in driving the jaw closing motion may be shorter than the length of travel of the actuation link 370 in the end effector 393 (compare arrow 602 in FIG. 6B with arrow 902 in FIG. 10B, which is described further below).

FIGS. 10A-10E illustrates states of the end effector 493 that are analogous to the states illustrated in FIGS. 6A-7E. Accordingly, the description of the states illustrated in FIGS. 6A-6E is applicable, mutatis mutandis, to the states illustrated in in FIGS. 6A-6E and duplicative description below is omitted.

FIG. 10A illustrates a first state in which jaw mechanism 450 is open. In this state the actuation link 470 is at a proximal position A₁, the moveable component 480 (comprising a cutting blade 481 in this embodiment) is at a first retracted position B₁, and the jaw members 451 and 452 are in open positions.

FIG. 10B illustrates a second state in which the jaw mechanism 450 is closed. This state is reached from the first state in FIG. 10A by driving the first actuation element 499 to move distally, as indicated by the arrow 901. As a result, the actuation link 470 is moved from the proximal position A₁ to the distal position A₂, as indicated by the arrow 902, which forces the jaw members 451 and 452 to move toward one another into closed positions, as indicated by the arrows 904. The moveable component 480 is also moved from the first retracted position B₁ to the second retracted position B₂, as indicated by the arrow 903.

FIG. 10C illustrates a third state in which the jaw mechanism 450 is closed and the moveable component 480 is extended. This state is reached from the second state in FIG. 10B by driving the second actuation element 498 to move distally, as indicated by the arrow 905. As a result, the moveable component 480 is moved from the second retracted position B₂ to the extended position B₃, as indicated by the arrow 906.

FIG. 10D illustrates a fourth state in which the jaw mechanism 3=450 is closed and the moveable component 480 is retracted. This state is reached from the third state in FIG. 10C by driving the second actuation element 498 to move proximally, as indicated by the arrow 907. As a result, the moveable component 480 is moved from the extended position B₃ back to the second retracted position B₂, as indicated by the arrow 908.

FIG. 10E illustrates a fifth state in which the jaw mechanism 450 is opened. This state is reached from the fourth state in FIG. 10C by driving the second actuation element 498 to move proximally, as indicated by the arrow 909. As a result, the moveable component 480 is moved from the second retracted position B₂ back to the first retracted position B₁, as indicated by the arrow 910. This pulls the actuation link 470 proximally along with the moveable component 480, thus moving the actuation link from the distal position A₂; back to the proximal position A₁, as indicated by the arrow 911. This motion of the actuation link 470 causes the jaw members 451 and 452 to move apart from one another toward their open positions, as indicated by the arrows 912.

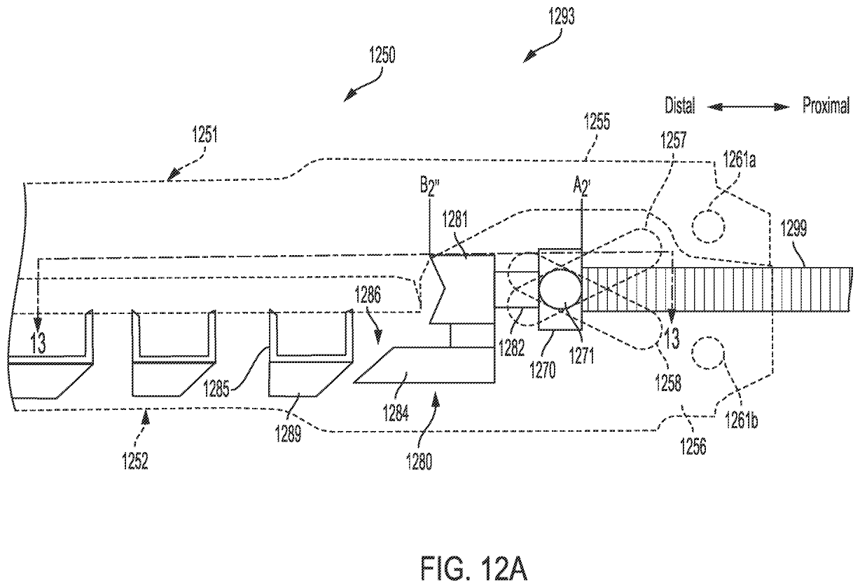

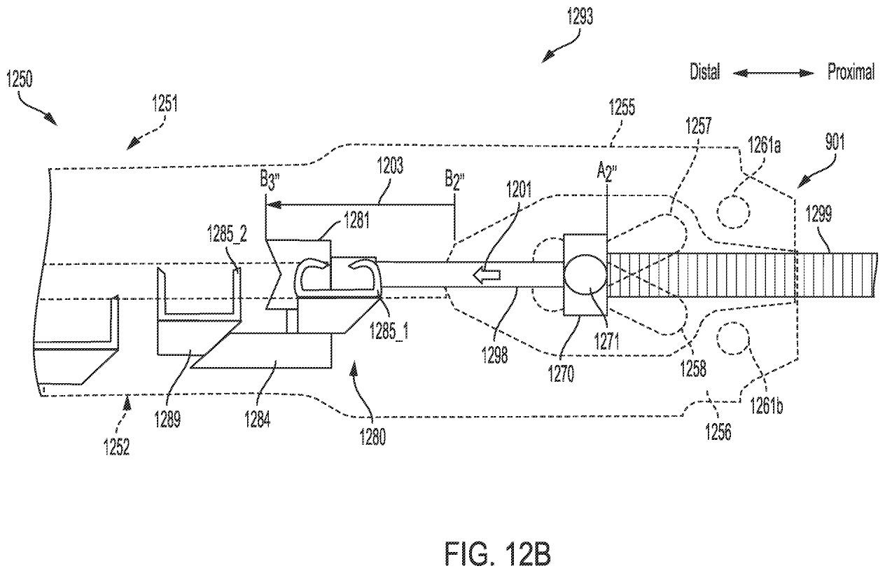

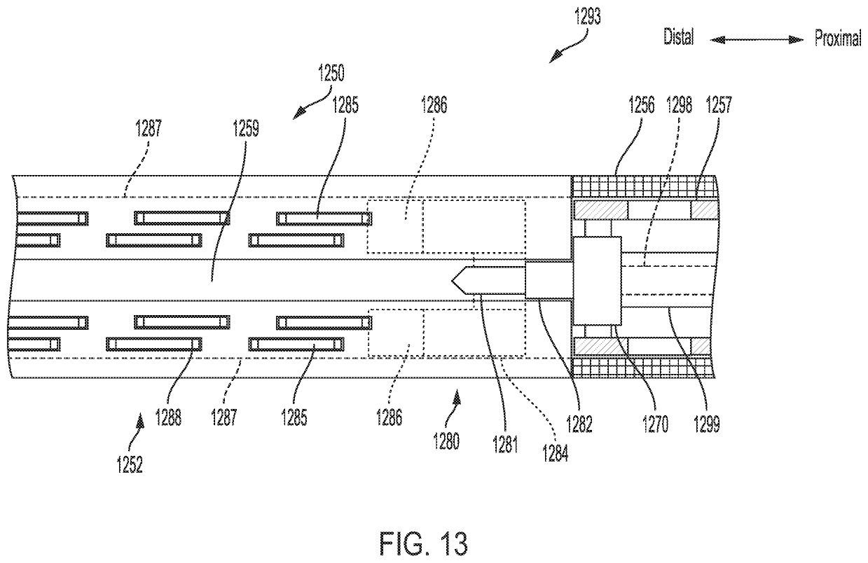

Turning now to FIGS. 12A-13, a third embodiment of an end effector 1293 is described in greater detail. The end effector 1293 may be used as the end effector 293. The end effector 1293 is similar to the end effector 493, except that the end effector 1293 is configured as a stapler, as described below. The end effector 1293 is also similar to the end effector 393, except that the end effector 1293 is configured as a stapler and also has two different jaw member pivot axis. Parts of the end effector 1293 that are similar to parts of the end effectors 393 and 493 are referred to using reference numerals having the same right-most digits (for example, jaw member 451 and jaw member 1251 are similar parts of the end effector 493 and the end effector 1293, respectively). The description of the parts of the end effectors 393 and 493 above are applicable to the similar parts of the end effector 1293, mutatis mutandis, and therefore duplicative descriptions of these similar components of the end effector 1293 are omitted below.

In FIGS. 12A and 12B, the jaw members 1251 and 1252 are made transparent and indicated by dashed lines. In FIG. 13, the jaw members 1252 is made transparent.

As noted above, the end effector 1293 is configured as a stapler. Thus, one of the jaw members 1251 and 1252 (e.g., jaw member 1252 in FIG. 12A) is configured to hold staples 1285 in an arrangement allowing the staples 1285 to be fired into material grasped between the jaw members 1251 and 1252, while the other one of the jaw members 1252 and 1251 (e.g., jaw member 1251 in FIG. 12A) is configured to act as an anvil to deform the staples 1285 and shape them into a closed configuration as the staples 1285 are fired. The staples 1285 may be fired through openings 1288 (see FIG. 13) in a surface of the jaw member 1251 or 1252 that contacts the grasped material. The staples 1285 may be held in corresponding receptacles (not illustrated) in the jaw member 1251 or 1252, or may be part of a removable cartridge (not illustrated) which may be received within the jaw member 1251 or 1252. Mechanisms for holding staples in a jaw member, such as the aforementioned receptacles and removable cartridges, would be familiar to one of ordinary skill in the art, and thus are not shown or described in greater detail.

Moreover, the movable component 1280 comprises a staple firing element 1284. The staple firing element 1284 comprises a ramped (sloped) surface 1286 that is arranged to come into contact with complementary ramped surfaces of pushers 1298 positioned below the staples 1285 as the movable component 1280 is translated distally. Contact between the ramped surface 1286 and the pushers 1298 causes the pushers 1298 to move in a direction substantially perpendicular to the direction of translation of movable component 1280 (i.e., an upward direction in FIGS. 12A and 12B), and this motion of the pushers 1298 forces the staples 1285 to move in the same direction), thereby firing the staples 1285. For example, in FIG. 12B one staple 1285_1 that has been fired by distal translation of the movable component 1280 is illustrated, and a second staple 1285_2 that is in the process of being fired is illustrated. A portion of the staple firing element 1284 which includes the ramped surface 1286 may be positioned below and adjacent to a surface of the jaw member 1252 that contacts the grasped material and may slide along a length of the jaw member 1252 through a channel 1287 within the jaw member 1252. The staples 1285 may be housed within the channel 1287.

In the illustrated embedment, the movable component 1280 also comprises a blade 1281. The blade 1281 translates along a track 1259, shown in FIG. 13. The blade 1281 may extend between the gap between the jaw members 1251 and 1252 in the closed state, as shown in FIGS. 12A and 12B. Thus, the blade 1281 may cut the grasped material as the movable component 1280 translates distally. In some embodiments, the blade 1281 may trail behind the ramped surface 1286 such that staples 1285 are fired into a portion of the grasped material prior to that portion being cut by the blade 1281. The blade 1281 and staple firing element 1284 may be coupled with the attachment portion 1282. In other embodiments, the movable component 1280 does not have a blade.

The staple firing element 1284 is coupled, directly or indirectly, to the attachment portion 1282, and thus is forced to move along with the attachment portion 1282 as the movable component 1280 is driven to translate. In the embodiment illustrated in FIGS. 12A-13, the staple firing element 1284 is coupled to the blade 1281 (e.g., a top portion of the staple firing element 1284 is coupled to a bottom portion of the blade 1281), and the blade 1281 is coupled to the attachment portion 1282. In other embodiments the staple firing element 1284 may be coupled directly to the attachment portion 1282. In some embodiments, the staple firing element 1284 is integrally connected with (i.e., part of the same monolithic body as) the blade 1281 and/or attachment portion 1282. In other embodiments, the staple firing element 1284, the blade 1281, and/or the attachment portion 1282 are separate parts that are joined together, for example by welding, solder, mechanical fasteners, adhesives, or any other joining technique.

As shown in FIG. 13, in some embodiments the staples 1285 may be arranged in two groups extending along a portion of the length of the jaw member 152, with the two groups being disposed on opposite sides of the track 1259. In FIG. 13 two groups of staples are shown with each of the groups comprising two rows of staples, but the specific number and arrangement of staples 1285 shown in FIG. 13 is not limiting and any number and arrangement of staples 1285 may be used.

Operation of the end effector 1293 will now be described, with reference to FIGS. 12A-12B. In FIGS. 12A and 12B, the jaw members 1251 and 1252 are shown as transparent and in dashed lines. FIG. 12A illustrates a state in which the jaw mechanism 1250 has been closed, which is analogous to the states illustrated in FIGS. 6B and 10B described above. The operations for bringing the end effector 1293 from an initial state to the closed state illustrated in FIG. 12A may be similar to those described above for bringing the end effectors 393 and 493 into the states illustrated in FIGS. 6B and 10B. After the jaw mechanism 1250 is closed in the state of FIG. 12A, the movable component 1280 may be fired or extended, transitioning the end effector to the state illustrated in FIG. 12B. The state illustrated in FIG. 12B is analogous to the states illustrated in FIGS. 6C and 10C described above. More specifically, the state illustrated in FIG. 12B is reached by driving the second actuation element 1298 to translate distally relative to the actuation link 1270 and relative to the jaw members 1251, 1252, as indicated by the arrow 1291, thus driving the movable component 1280 to translate distally from the second retracted position B₂″ (analogous to the positions B₂ and B₂′ described above) to an extended position B₃″ (analogous to the positions B₃ and B₃′ described above) as indicated by the arrow 1203. FIG. 12B illustrates the movable component 1280 in a partially extended state so as to show the movable component 1280 interacting with staples 1285, but in practice the movable component 1280 may be extended even farther distally along the jaw member 1252. The distal translation of the movable component 1280 causes the staple firing element 1284 thereof to collide with and fire one or more staples 1285 into a material grasped between the jaw members 1251 and 1252, as described above. The distal translation of the movable component 1280 may also cause the blade 1281 to cut the material grasped between the jaw members 1251 and 1252, as described above. After firing (extending) the movable component 1280, the movable component 1280 may be retracted and the jaw mechanism 1250 may be opened. Operations for retracting the movable component 1280 and opening the jaw mechanism 1250 may be similar to those described above with respect to the end effectors 393 and 493, and thus are not illustrated or described again herein.

Turning now to FIG. 11, an embodiment of a computer-assisted instrument control system 100 for remote control of instruments will be described. FIG. 11 is a schematic block diagram of the computer-assisted instrument control system 100 for remote control of instruments. The system 100 comprises a manipulator assembly 110, a control system 106, and a user input and feedback system 104. The system 100 may also include an auxiliary system 108. These components of the system 100 are described in greater detail blow.

The manipulator assembly 110 comprises one or more manipulators 114. FIG. 11 illustrates three manipulators 114, but any number of manipulators 114 may be included. In the embodiment of FIG. 11, each manipulator 114 comprises a kinematic structure of two or more links 115 coupled together by one or more joints 116. The joints 116 may impart various degrees of freedom of movement to the manipulator 114, allowing the manipulator 114 to be moved around a workspace. For example, some joints 116 may provide for rotation of links 115 relative to one another, other joints 116 may provide for translation of links 115 relative to one another, and some may provide for both rotation and translation. Some or all of the joints 116 may be powered joints, meaning a powered drive element may control movement of the joint 116 through the supply of motive power. Such drive elements may comprise, for example, electric motors, pneumatic or hydraulic actuators, etc. Additional joints 116 may be unpowered joints. In addition to drive elements that control the joints 116, the manipulator 114 may also include drive elements (not illustrated) that drive inputs of the instrument 102 to control operations of the instrument, such as moving an end-effector of the instrument, opening/closing jaws, driving translating and/or rotating components, etc. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below. FIG. 10 illustrates each manipulator 114 as having two links 115 and one joint 116, but in practice a manipulator may include more links 115 and more joints 116, depending on the needs of the system 100. The more links 115 and joints 116 are included, the greater the degrees of freedom of movement of the manipulator 114.

Each manipulator 114 may be configured to support and/or operate one or more instruments 102. In some examples the instruments 102 may be fixedly coupled to the manipulator 114, while in other examples one of the links 115 may be configured to have one or more separate instruments 102 removably coupled thereto. The instruments 102 may include any tool or instrument, including for example industrial instruments and medical instruments (e.g., surgical instruments, imaging instruments, diagnostic instruments, therapeutic instruments, etc.). The instrument 202 described above may be used as any one of the instruments 102.

The system 100 can also include a user input and feedback system 104 operably coupled to the control system 106. The user input and feedback system 104 comprises one or more input devices to receive input control commands to control operations of the manipulator assembly 110. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 104 may also include feedback devices, such as a display device (not shown) to display images (e.g., images of the worksite as captured by one of the instruments 102), haptic feedback devices, audio feedback devices, other graphical user interface forms of feedback, etc.

The control system 106 may control operations of the system 100. In particular, the control system 106 may send control signals (e.g., electrical signals) to the manipulator assembly 110 to control movement of the joints 116 and to control operations of the instruments 102 (e.g., through drive interfaces at the manipulators 114). In some embodiments, the control system 106 may also control some or all operations of the user input and feedback system 104, the auxiliary system 108, or other parts of the system 100. The control system 106 may include an electronic controller to control and/or assist a user in controlling operations of the manipulator assembly 110. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

As noted above, differing degrees of user control versus autonomous control may be utilized in the system 100, and embodiments disclosed herein may encompass fully user-controlled systems, fully autonomously-controlled systems, and systems having any combination of user and autonomous control. For operations that are user-controlled, the control system 106 generates control signals in response to receiving a corresponding user input command via the user input and feedback system 104. For operations that are autonomously controlled, the control system 106 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled—for example, a user input command may initiate performance of a sequence of events, and then the control system 106 may perform various operations associated with that sequence without needing further user input.

The auxiliary system 108 may comprise various auxiliary devices that may be used in operation of the system 100. For example, the auxiliary system 108 may include power supply units, auxiliary function units (e.g., functions such as irrigation, evacuation, energy supply, illumination, sensors, imaging, etc.). As one example, in a system 100 for use in a medical procedure context, the auxiliary system 108 may comprise a display device for use by medical staff assisting a procedure, while the user operating the input devices may utilize a separate display device that is part of the user input and feedback system 104. As another example, in a system 100 for use in a medical context, the auxiliary system 108 may comprise flux supply units that provide surgical flux (e.g., electrical power) to instruments 102. An auxiliary system 108 as used herein may thus encompass a variety of components and does not need to be provided as an integral unit.

As noted above, one or more instruments 102 can be mounted to the manipulator 114. In some embodiments, an instrument carriage physically supports the mounted instrument 102 and has one or more actuators (not illustrated) to provide driving forces to the instrument 102 to control operations of the instrument 102. The actuators may provide the driving forces by actuating drive outputs (not illustrated), such as rotary disc outputs, joggle outputs, linear motion outputs, etc. The drive outputs may interface with and mechanically transfer driving forces to corresponding drive inputs of the instrument 102 (directly, or via intermediate drive outputs, which may be part of a sterile instrument adaptor (ISA) (not illustrated)). The ISA may be placed between the instrument 102 and the instrument carriage to maintain sterile separation between the instrument 102 and the manipulator 114. The instrument carriage may also comprise other interfaces (not illustrated), such as electrical interfaces to provide and/or receive electrical signals to/from the instrument 102.

The embodiments described herein (including the system 100, instrument 202, end effector 393, and end effector 493 described above) may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, including, but not limited to, sensing, inspecting, and/or manipulating non-tissue work pieces. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. Further, the terminology used herein to describe aspects of the invention, such as spatial and relational terms, is chosen to aid the reader in understanding example embodiments of the invention but is not intended to limit the invention. For example, spatially terms-such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

As used herein, “proximal” and “distal” are spatial/directional terms that describe locations or directions based on their relationship. In the context of the present disclosure, the directions proximal and distal are labeled relative to the instrument in various figures, with proximal describing the direction along the instrument toward the force transmission system and distal describing the direction along the instrument toward the end effector. As such, the proximal and distal directions are not fixed in space, but rather are used herein to describe different end portions of the instrument itself regardless of its specific orientation in space.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances. Furthermore, the range of variation also includes at least variations that are within +5% of the stated value, property, or relationship. Thus, for example, a line or surface may be considered as being “approximately parallel” to a reference line or surface if any one of the following is true: the smallest angle between the line/surface and the reference is less than or equal to 4.5° (i.e., 5% of) 90°, the angle is less than or equal to manufacturing or other tolerances typical in the art, or the line/surface as constituted is functionally equivalent to the line/surface if it had been perfectly parallel.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. In some instances, well-known structures, systems, and techniques have not been shown or described in detail in order not to obscure the embodiments. Changes may be made in the elements described herein without departing from the scope of the present teachings and following claims.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

The following listing of claims replaces all prior versions and listings of claims:

1. A medical instrument comprising:
a shaft comprising a distal end portion and a proximal end portion;
an end effector coupled to the distal end portion of the shaft and comprising:
a jaw mechanism; and
a movable element translatable relative to the jaw mechanism;
a first actuation element operably coupled to the end effector and translatable relative to the shaft; and
a second actuation element operably coupled to the end effector and translatable relative to the shaft, the second actuation element being translatable relative to the first actuation element within at least a first range of motion of the second actuation element;
wherein:
translation of the first actuation element in a first direction relative to the shaft drives closing of the jaw mechanism;
translation of the second actuation element in a second direction relative to the shaft, within a second range of motion of the second actuation element drives opening of the jaw mechanism, the second direction being opposite from the first direction; and
in the closed state of the jaw mechanism, translation of the second actuation element relative to the shaft in the first and second directions within the first range of motion drives translation of the movable element relative to the jaw mechanism.

2. The medical instrument of claim 1, wherein the first actuation element comprises a flexible push member.

3. The medical instrument of claim 2, wherein the second actuation element is routed through an interior channel of the first actuation element.

4. The medical instrument of claim 1, wherein the movable component comprises a cutting element.

5. The medical instrument of claim 1, wherein:
the first direction is a distal direction pointing from the proximal end portion of the shaft toward the distal end portion of the shaft; and
the second direction is a proximal direction pointing from the distal end portion of the shaft toward the proximal end portion of the shaft.

6. The medical instrument of claim 1, wherein:
the end effector further comprises an actuation link that is translatable in the first direction to cause closing of the jaw mechanism and translatable in the second direction to cause opening of the jaw mechanism;
the first actuation element is configured to, when translated in the first direction, push the actuation link in the first direction; and
the second actuation element is configured to, when translated in the second direction, pull the actuation link in the second direction.

7. The medical instrument of claim 6, wherein:
in the first range of motion of the second actuation element, the second actuation element is translatable relative to the actuation link in the first and second directions; and
in the second range of motion of the second actuation element, translation of the second actuation element in the second direction causes the movable component to contact and pull the actuation link in the proximal direction.

8. The medical instrument of claim 6, wherein
the second actuation element extends through the actuation link via a hole in the actuation link and is coupled to the movable component; and
the movable component is larger in at least one lateral dimension than a diameter of the hole in the actuation link.

9. The medical instrument of claim 1, wherein:
the end effector further comprises a clevis; and
the jaw mechanism comprises first and second jaw members pivotably coupled to the clevis to pivot about a same pivot axis.

10. The medical instrument of claim 1, wherein:
the end effector further comprises a clevis; and
the jaw mechanism comprises first and second jaw members respectively pivotably coupled to the clevis to pivot about two differing pivot axes.

11. (canceled)

12. (canceled)

13. (canceled)

14. (canceled)

15. The medical instrument of claim 1, further comprising:
an articulable structure coupling the end effector to the shaft, the articulable structure configured to provide one or more degrees of freedom of motion to the end effector relative to the shaft;
wherein the first actuation element comprises a flexible push member extending through the articulable structure and flexible along at least the one or more degrees of freedom of motion; and
wherein the second actuation element comprises a flexible push-pull member extending through the articulable structure and flexible along at least the one or more degrees of freedom of motion.

16. (canceled)

17. (canceled)

18. The medical instrument of claim 15, wherein the flexible push member comprises a push coil or a laser cut tube and the flexible push-pull member comprises a cable, wire, or filament.

19. (canceled)

20. (canceled)

21. (canceled)

22. The medical instrument of claim 15, wherein the first actuation element further comprises a non-flexible push member coupled to the flexible push member.

23. (canceled)

24. The medical instrument of claim 1, further comprising a force transmission system coupled to the proximal end portion and operably coupled to the first and second actuation elements, the force transmission system comprising an interface comprising first and second drive inputs coupled to and configured to drive the first and second actuation elements, respectively.

25. A system comprising:
a manipulator comprising an interface configured to engage with the medical instrument of claim 1 and transfer driving forces to the first and second actuation elements.

26. The system of claim 25, further comprising:
a control system operably coupled to the manipulator to control supply of the driving forces to the medical instrument, the control system configured to:
close the jaw mechanism of the medical instrument by translating the first actuation element to push an actuation link of the end effector to translate in the first direction;
in the closed state of the jaw mechanism, extend and retract the movable component relative to the jaw mechanism by translating the second actuation element relative to the actuation link within [a] the first range of motion while the first actuation element is stationary relative to the actuation link;
open the jaw mechanism by driving the second actuation element to translate in the second direction within the second range of motion to pull the actuation link to translate in the second direction.

27-42. (canceled)

43. A method of operating an instrument comprising a shaft and an end effector coupled to the shaft, the method comprising:
closing a jaw mechanism of the end effector by translation of a first actuation element in a first direction relative to the shaft;
in a closed state of the jaw mechanism, translating a movable component of the end effector in the first direction over a first distance relative to the jaw mechanism, by translating a second actuation element in the first direction relative to the shaft within a first range of motion while the first actuation element is stationary relative to the shaft;
in the closed state of the jaw mechanism, translating the movable component in a second direction back over the first distance relative to the jaw mechanism, by translating the second actuation element in the second direction relative to the shaft within the first range of motion while the first actuation element is stationary relative to the shaft, the second direction being opposite the first direction; and
opening the jaw mechanism by translating the movable component in the second direction relative to the shaft over a second distance extending in the second direction from the first distance by translating the second actuation element in the second direction within a second range of motion.

44. The method of claim 43, wherein:
closing the jaw mechanism comprises pushing an actuation link operably coupled to the jaw mechanism in the first direction by the translation of the first actuation element in the first direction; and
opening the jaw mechanism comprises pulling the actuation link in the second direction by the translation of the moveable component in the second direction over the second distance.

45. (canceled)

46. The method of claim 44, wherein
translating the second actuation element within the first range of motion comprises translating the second actuation element relative to the actuation link; and
pulling the actuation link in the second direction comprises contacting and pulling the actuation link with the movable component.

47. (canceled)

48. The method of claim 18, wherein:
opening the jaw mechanism comprises translating both the first and second actuation elements in the second direction relative to the shaft with the first actuation element applying a first force to the actuation link and the second actuation element applying a second force to the actuation link via the movable component; and
the first force is smaller than the second force.

49-52. (canceled)
